(19) United States
(12) Patent Application Publication
(10) Pub. No.: US 2024/0191179 A1
(43) Pub. Date: Jun. 13, 2024

(54) METHOD FOR SUPPRESSING PRODUCTION OF DEGRADATION PRODUCTS

(71) Applicant: KYOWA KIRIN CO., LTD., Tokyo (JP)

(72) Inventors: Tsuyoshi YAMAGUCHI, Tokyo (JP); Mie FUKUDA, Tokyo (JP); Hiroko ISHIKAWA, Tokyo (JP); Ryuma NAGANO, Tokyo (JP); Toshiyuki SUZAWA, Tokyo (JP)

(73) Assignee: KYOWA KIRIN CO., LTD., Tokyo (JP)

(21) Appl. No.: 18/287,750

(22) PCT Filed: Apr. 22, 2022
(86) PCT No.: PCT/JP2022/018636

§ 371 Date: Oct. 20, 2023

(30) Foreign Application Priority Data
Apr. 23, 2021 (JP) 2021-073666

(51) Int. Cl. C12N 5/00

(52) U.S. Cl. CPC C12N 5/0018; C12N 2500/32; C12N 2500/46; C12N 2501/999

(57) ABSTRACT

An object of the present invention is to provide a culture method by which an LMWS amount is minimized while high productivity of a target protein is maintained. The present invention relates to a method for preventing generation of a degradation product (a low molecular weight species: LMWS) of a target protein. The method includes: a means for, in a cell culture process for producing the target protein at a high concentration in a culture medium, removing a reactive oxygen species in the culture medium.

### TECHNICAL FIELD

The present invention relates to a method for reducing an amount of a degradation product secondarily produced during recombinant protein expression.

### BACKGROUND ART

With the development of a gene recombination technique in recent years, a biopharmaceutical product, including a protein pharmaceutical product such as an antibody, is now widely available. The protein pharmaceutical product is produced by using a producing cell prepared by introducing, into a host cell such as Escherichia coli, a yeast, an insect cell, a plant cell, and an animal cell, an expression vector containing a nucleotide sequence encoding a recombinant protein (hereinafter also referred to as a “target protein” in order to distinguish from a protein that is translated and secreted from the same cell based on an endogenous gene).

In a process commonly used as a process for producing a protein pharmaceutical product, first, producing cells are cultured under appropriate conditions to secrete a target protein into a culture medium. The culture medium containing the target protein is subjected to purification after removal of unnecessary producing cells.

Among the protein pharmaceutical products, unlike a biologically active substance, an antibody pharmaceutical product requires a large dosage due to an action mechanism thereof, and productivity in a producing process is important. An effort to improve productivity in a culture process is widely performed, but there is a problem in quality variation such as an increase in an amount of a polymer accompanying the improvement in productivity (Non-Patent Literature 1).

In the culture process, an antibody in the culture medium may be degraded due to a chemical reaction with a medium component, dissolved oxygen, or the like or activity of various enzymes derived from producing cells. Even when the culture medium containing the target antibody is purified, a degradation product (a low molecular weight species: LMWS) thereof may remain depending on a purification degree thereof. A content of the LMWS in the antibody pharmaceutical product is generally required to be controlled so as to satisfy certain acceptable standards as critical quality attributes (CQA) (Non-Patent Literature 2).

Examples of the cause of antibody degradation include exposure to extremely high or low pH conditions, reduction of an antibody, and a radical chain reaction caused by reactive oxygen (Non-Patent Literature 3, Patent Literature 1). It is known that a peptide bond in the vicinity of a disulfide bond for connecting an antibody H chain and L chain is degraded due to a radical chain reaction caused by reactive oxygen, and an L chain and an HHL body (with one L chain detached), Fab, or the like is produced as a degradation product (Non-Patent Literatures 4 and 5).

Examples of a method for reducing an LMWS amount in a culture process include a method using S-sulfocysteine instead of cysteine which is one of medium components (Non-Patent Literature 6). However, at present, there are very few methods for reducing the LMWS amount by focusing on other medium components in the culture process.

### CITATION LIST

### Patent Literature

Patent Literature 1: EP Patent No. 2586788 specification

### Non-Patent Literature

Non-Patent Literature 1: “Biotechnology and Bioengineering”, 2018, Vol. 115, pp. 126-138
Non-Patent Literature 2: “Journal of Pharmaceutical and Biomedical Analysis”, 2020, Vol. 184, 113166
Non-Patent Literature 3: “mAbs”, 2011, Vol. 3, pp. 253-263
Non-Patent Literature 4: “The Journal of Biological Chemistry”, 2009, Vol. 284, pp. 35390-35402
Non-Patent Literature 5: “The Journal of Biological Chemistry”, 2011, Vol. 286, pp. 24674-24684
Non-Patent Literature 6: “mAbs”, 2017, Vol. 9, pp. 889-897

### SUMMARY OF INVENTION

### Technical Problem

The present inventors have newly found that the LMWS tends to increase when the antibody productivity is improved. From the study of the present inventors, it was presumed that the cause of the LMWS increase is reactive oxygen. By improving the antibody productivity, a cell activity degree is increased, resulting in production of a large amount of reactive oxygen. It is considered that the radical chain reaction caused by the reactive oxygen is involved in the LMWS increase.

Accordingly, an object of the present invention is to provide a culture method by which an LMWS amount is minimized while high productivity of a target protein is maintained.

### Solution to Problem

The present inventors have extensively studied a culture method by which an LMWS amount is minimized while high productivity of a target protein is maintained. As a result, the present inventors have found out a culture method by which an LMWS amount is minimized while high productivity of a target protein is maintained by applying a method for removing a reactive oxygen species in a culture medium in a culture process, thereby completing the invention.

That is, the present invention relates to the following. 1. A method for preventing generation of a degradation product (a low molecular weight species: LMWS) of a target protein, the method including: a means for, in a cell culture process for producing the target protein at a high concentration in a culture medium, removing a reactive oxygen species in the culture medium. 2. The method according to the above 1, wherein the target protein is an antibody, and an antibody concentration in the culture medium at the end of the cell culture is 4.0 g/L or more.

3. The method according to the above 1 or 2, wherein a generation amount of the LMWS is reduced as compared with a cell culture process not including the means for removing the reactive oxygen species.

4. The method according to any one of the above 1 to 3, wherein the generation amount of the LMWS is reduced by 0.1% or more as compared with the cell culture process not including the means for removing the reactive oxygen species.

5. The method according to any one of the above 1 to 4, wherein the generation amount of the LMWS is reduced by 0.5% or more as compared with the cell culture process not including the means for removing the reactive oxygen species.

6. The method according to any one of the above 1 to 5, wherein the generation amount of the LMWS is reduced by 1.0% or more as compared with the cell culture process not including the means for removing the reactive oxygen species.

7. The method according to any one of the above 1 to 6, wherein the means for removing the reactive oxygen species is at least one selected from the following (a) to (e):
(a) adding an antioxidant to a medium used for the cell culture,
(b) making a cystine or cystine analogue concentration in the culture medium at the end of the cell culture 1.90 mmol/L or less,
(c) making a copper concentration in the culture medium at the end of the cell culture 20.0 μmol/L or less,
(d) adding a chelating compound to a medium used for the cell culture, and (e) making a pH of a feed medium used for the cell culture 8.0 or more.

8. The method according to the above 7, wherein in the (a), the antioxidant is a catechin analogue.

9. The method according to the above 8, wherein a catechin analogue concentration in the culture medium at the end of the cell culture is 50 μmol/L or more.

10. The method according to the above 9, wherein the catechin analogue is epigallocatechin gallate, and an epigallocatechin gallate concentration in the culture medium at the end of the cell culture is 50 μmol/L to 300 μmol/L.

11. The method according to the above 9 or 10, wherein the catechin analogue is epigallocatechin gallate, and the epigallocatechin gallate concentration in the culture medium at the end of the cell culture is 50 μmol/L to 250 μmol/L.

12. The method according to the above 9, wherein the catechin analogue is catechin hydrate, and a catechin hydrate concentration in the culture medium at the end of the cell culture is 50 μmol/L to 450 μmol/L.

13. The method according to the above 9 or 12, wherein the catechin analogue is catechin hydrate, and the catechin hydrate concentration in the culture medium at the end of the cell culture is 100 μmol/L to 400 μmol/L.

14. The method according to the above 7, wherein in the (b), the cystine or cystine analogue concentration in the culture medium at the end of the cell culture is 0.50 mmol/L to 1.90 mmol/L.

15. The method according to the above 7 or 14, wherein in the (b), the cystine or cystine analogue concentration in the culture medium at the end of the cell culture is 1.00 mmol/L to 1.90 mmol/L.

16. The method according to the above 7, wherein in the (c), the copper concentration in the culture medium at the end of the cell culture is 0.10 μmol/L to 20.0 μmol/L.

17. The method according to the above 7 or 16, wherein in the (c), the copper concentration in the culture medium at the end of the cell culture is 0.10 μmol/L to 0.50 μmol/L.

18. The method according to the above 7, wherein in the (d), the chelating compound is EDTA or citric acid.

19. The method according to the above 7 or 18, wherein in the (d), the chelating compound is citric acid, and a citric acid concentration in the culture medium at the end of the cell culture is 1.50 mmol/L to 8.00 mmol/L.

20. The method according to any one of the above 7, 18, and 19, wherein in the (d), the chelating compound is citric acid, and the citric acid concentration in the culture medium at the end of the cell culture is 1.80 mmol/L to 6.50 mmol/L.

21. The method according to the above 7, wherein in the (e), the pH of the feed medium is 8.0 to 9.0.

22. The method according to the above 7 or 21, wherein in the (e), the pH of the feed medium is 8.0 to 8.6.

23. A method for producing a target protein containing a reduced LMWS amount at a high concentration in a culture medium, the method including: a means for removing a reactive oxygen species in the culture medium.

24. The method according to the above 23, wherein the target protein is an antibody, and an antibody concentration in the culture medium at the end of the cell culture is 4.0 g/L or more.

25. The method according to the above 23 or 24, wherein a generation amount of the LMWS is reduced as compared with a cell culture process not including the means for removing the reactive oxygen species.

26. The method according to any one of the above 23 to 25, wherein the generation amount of the LMWS is reduced by 0.1% or more as compared with the cell culture process not including the means for removing the reactive oxygen species.

27. The method according to any one of the above 23 to 26, wherein the generation amount of the LMWS is reduced by 0.5% or more as compared with the cell culture process not including the means for removing the reactive oxygen species.

28. The method according to any one of the above 23 to 27, wherein the generation amount of the LMWS is reduced by 1.0% or more as compared with the cell culture process not including the means for removing the reactive oxygen species.

29. The method according to any one of the above 23 to 28, wherein the means for removing the reactive oxygen species is at least one selected from the following (a) to (e):
(a) adding an antioxidant to a medium used for the cell culture,
(b) making a cystine or cystine analogue concentration in the culture medium at the end of the cell culture 1.90 mmol/L or less,
(c) making a copper concentration in the culture medium at the end of the cell culture 20.0 μmol/L or less,
(d) adding a chelating compound to a medium used for the cell culture, and
(e) making a pH of a feed medium used for the cell culture 8.0 or more.

30. The method according to the above 29, wherein in the (a), the antioxidant is a catechin analogue.

31. The method according to the above 30, wherein a catechin analogue concentration in the culture medium at the end of the cell culture is 50 μmol/L or more.

32. The method according to the above 31, wherein the catechin analogue is epigallocatechin gallate, and an epigallocatechin gallate concentration in the culture medium at the end of the cell culture is 50 μmol/L to 300 μmol/L.

33. The method according to the above 31 or 32, wherein the catechin analogue is epigallocatechin gallate, and the epigallocatechin gallate concentration in the culture medium at the end of the cell culture is 50 μmol/L to 250 μmol/L.

34. The method according to the above 31, wherein the catechin analogue is catechin hydrate, and a catechin hydrate concentration in the culture medium at the end of the cell culture is 50 μmol/L to 450 μmol/L.

35. The method according to the above 31 or 34, wherein the catechin analogue is catechin hydrate, and the catechin hydrate concentration in the culture medium at the end of the cell culture is 100 μmol/L to 400 μmol/L.

36. The method according to the above 29, wherein in the (b), the cystine or cystine analogue concentration in the culture medium at the end of the cell culture is 0.50 mmol/L to 1.90 mmol/L. 37. The method according to the above 29 or 36, wherein in the (b), the cystine or cystine analogue concentration in the culture medium at the end of the cell culture is 1.00 mmol/L to 1.90 mmol/L. 38. The method according to the above 29, wherein in the (c), the copper concentration in the culture medium at the end of the cell culture is 0.10 μmol/L to 20.0 μmol/L.

39. The method according to the above 29 or 38, wherein in the (c), the copper concentration in the culture medium at the end of the cell culture is 0.10 μmol/L to 0.50 μmol/L. 40. The method according to the above 29, wherein in the (d), the chelating compound is EDTA or citric acid. 41. The method according to the above 29 or 40, wherein in the (d), the chelating compound is citric acid, and a citric acid concentration in the culture medium at the end of the cell culture is 1.50 mmol/L to 8.00 mmol/L. 42. The method according to any one of the above 29, 40, and 41, wherein in the (d), the chelating compound is citric acid, and the citric acid concentration in the culture medium at the end of the cell culture is 1.80 mmol/L to 6.50 mmol/L. 43. The method according to the above 29, wherein in the (e), the pH of the feed medium is 8.0 to 9.0.

44. The method according to the above 29 or 43, wherein in the (e), the pH of the feed medium is 8.0 to 8.6.

### Advantageous Effects of Invention

According to the method of the present invention, by including a means for, in a cell culture process for producing a target protein at a high level in a culture medium, removing a reactive oxygen species in the culture medium, generation of an LMWS can be effectively prevented while high productivity of the target protein can be maintained.

### BRIEF DESCRIPTION OF DRAWINGS

FIG. 1 shows that application of a highly-productive process increases an LMWS content after an end of culture, in which Mab A, Mab B, and Mab C respectively represent a monoclonal antibody A, a monoclonal antibody B, and a monoclonal antibody C; “initial” represents an initial process, and “highly-productive” represents a highly-productive process; Titer represents an antibody concentration in a culture supernatant, which is shown by a white bar graph with a unit of g/L on a vertical axis; and LMWS represents a degradation product and shows a proportion of the LMWS in an antibody collecting liquid after affinity purification obtained as a result of capillary electrophoresis, which is represented by a black bar graph with a unit of % on the vertical axis.

FIG. 2A shows an electropherogram obtained by capillary electrophoresis of an antibody (Mab A) produced in an initial process, and FIG. 2B is an electropherogram obtained by capillary electrophoresis of an antibody (Mab A) produced in a highly-productive process 1, in which the numbers 1 to 11 in the graph represent peak numbers.

FIG. 3 shows an electropherogram obtained by capillary electrophoresis of a purified antibody with hydrogen peroxide added, in which +20 mmol/L H₂O₂ represents an electropherogram of the purified antibody (Mab A) with hydrogen peroxide added at a final concentration of 20 mmol/L, +50 mmol/L H₂O₂ represents an electropherogram of the purified antibody with hydrogen peroxide added at a final concentration of 50 mmol/L, +20 mmol/L H₂O₂, 20 mmol/L EDTA represents an electropherogram of the purified antibody with hydrogen peroxide and EDTA added at a final concentration of 20 mmol/L each, no spike is a negative control where the purified antibody is not spiked, and each molecular species of the LMWS is schematically shown.

FIG. 4A shows LMWS contents after an end of flask culture of Mab A-producing CHO cells in a highly-productive process 2 with different concentrations of epigallocatechin gallate added to a medium, FIG. 4B shows LMWS contents after an end of flask culture of Mab C-producing CHO cells in a highly-productive process with different concentrations of epigallocatechin gallate added to a medium, and in FIGS. 4A and 4B, a horizontal axis represents an epigallocatechin gallate concentration (μmol/L) in a culture medium at the end of the culture, and a vertical axis represents Titer (g/L) and a proportion (%) of the LMWS in an antibody collecting liquid after affinity purification obtained as a result of capillary electrophoresis, and a bar graph represents the proportion of the LMWS in the antibody collecting liquid after the affinity purification, and a line graph represents Titer.

FIG. 5 shows LMWS contents after an end of flask culture of Mab A-producing CHO cells in the highly-productive process 2 with different concentrations of catechin hydrate added to a medium, in which a horizontal axis represents a catechin hydrate concentration (μmol/L) in a culture medium at the end of the culture, and a vertical axis represents Titer (g/L) and a proportion (%) of the LMWS in the antibody collecting liquid after affinity purification obtained as a result of capillary electrophoresis, and a bar graph represents the proportion of the LMWS in the antibody collecting liquid after the affinity purification, and a line graph represents Titer.

FIG. 6A shows a transition of viable cell density when reactor-culturing Mab A-producing CHO cells in the highly-productive process 2, in which a horizontal axis represents a culture time (day), and a vertical axis represents a viable cell density (×10⁵ cells/mL), FIG. 6B shows a transition of viability when reactor-culturing Mab A-producing CHO cells in the highly-productive process 2, in which a horizontal axis represents a culture time (day), and a vertical axis represents the viability (%), FIG. 6C shows a transition of Titer when reactor-culturing Mab A-producing CHO cells in the highly-productive process 2, in which a horizontal axis represents a culture time (day), and a vertical axis represents Titer (g/L), and in FIGS. 6A to 6C, a ▪(black square) mark (Control) represents a control condition, a ▴(black triangle) mark (EGCG) represents an epigallocatechin gallate addition condition, and a ●(black circle) mark (Catechin) represents a catechin hydrate addition condition, and an error bar represents a standard deviation at n=3, FIG. 6D shows LMWS contents after an end of reactor culture of Mab A-producing CHO cells in the highly-productive process 2 under a condition in which epigallocatechin gallate or catechin hydrate is added or a condition in which epigallocatechin gallate and catechin hydrate are not added, in FIG. 6D, a vertical axis represents a proportion (%) of the LMWS in an antibody collecting liquid after affinity purification obtained as a result of capillary electrophoresis, Control represents a control condition, EGCG represents an epigallocatechin gallate addition condition, and Catechin represents a catechin hydrate addition condition, and an error bar represents a standard deviation at n=3,

FIG. 7 shows LMWS contents after an end of culture at different cystine concentrations in a culture medium, in which a horizontal axis represents the cystine concentration (mmol/L) in the culture medium at the end of the culture, and a vertical axis represents Titer (g/L) and a proportion (%) of the LMWS in an antibody collecting liquid after affinity purification obtained as a result of capillary electrophoresis, and a bar graph represents the proportion of the LMWS in the antibody collecting liquid after the affinity purification, and a line graph represents Titer.

FIG. 8 shows LMWS contents after an end of culture at different copper concentrations in a culture medium, in which a horizontal axis represents the copper concentration (μmol/L) in the culture medium at the end of the culture, and a vertical axis represents Titer (g/L) and a proportion (%) of the LMWS in an antibody collecting liquid after affinity purification obtained as a result of capillary electrophoresis, and a bar graph represents the proportion of the LMWS in the antibody collecting liquid after the affinity purification, and a line graph represents Titer.

FIG. 9 shows LMWS contents after an end of culture at different amounts of citric acid added to a medium, in which a horizontal axis represents the citric acid concentration (mmol/L) in the culture medium at the end of the culture, and a vertical axis represents Titer (g/L) and a proportion (%) of the LMWS in an antibody collecting liquid after affinity purification obtained as a result of capillary electrophoresis, and a bar graph represents the proportion of the LMWS in the antibody collecting liquid after the affinity purification, and a line graph represents Titer.

FIG. 10 shows LMWS contents after an end of culture of Mab B-producing CHO cells at different pH in a feed medium, in which a horizontal axis represents the pH in the feed medium, and a vertical axis represents Titer (g/L) and a proportion (%) of the LMWS in an antibody collecting liquid after affinity purification obtained as a result of capillary electrophoresis, and a bar graph represents the proportion of the LMWS in the antibody collecting liquid after the affinity purification, and a line graph represents Titer.

FIG. 11 shows LMWS contents after an end of culture of Mab C-producing CHO cells at different pH in a feed medium, in which a horizontal axis represents the pH in the feed medium, and a vertical axis represents Titer (g/L) and a proportion (%) of the LMWS in an antibody collecting liquid after affinity purification obtained as a result of capillary electrophoresis, and a bar graph represents the proportion of the LMWS in the antibody collecting liquid after the affinity purification, and a line graph represents Titer.

### DESCRIPTION OF EMBODIMENTS

The present invention relates to a method for preventing generation of an LMWS of a target protein in a cell culture process for producing the target protein at a high concentration in a culture medium. The method includes a means for removing a reactive oxygen species in the culture medium.

Specifically, the cell culture process for producing the target protein at a high concentration in a culture medium refers to a process in which cells are cultured using a medium to produce the target protein at a high concentration in a culture medium.

### <Target Protein>

The target protein is preferably a protein derived from a eukaryotic cell, more preferably a protein derived from an animal cell, and examples thereof include a protein derived from a mammalian cell. The protein may have any structure as long as it includes the target protein and has a desired activity. For example, the protein may be an artificially modified protein such as a fusion protein fused with another protein, or a protein consisting of partial fragments.

Specific examples of the protein include a glycoprotein and an antibody.

Specific examples of the glycoprotein include erythropoietin (EPO) [J. Biol. Chem., 252, 5558 (1977)], thrombopoietin (TPO) [Nature, 369 533 (1994)], a tissue-type plasminogen activator, pro-urokinase, thrombomodulin, antithrombin III, protein C, protein S, blood coagulation factor VII, blood coagulation factor VIII, blood coagulation factor IX, blood coagulation factor X, blood coagulation factor XI, blood coagulation factor XII, a prothrombin complex, fibrinogen, albumin, gonadotropin, thyroid-stimulating hormone, an epidermal growth factor (EGF), a hepatocyte growth factor (HGF), a keratinocyte growth factor, activin, an osteogenic factor, a stem cell factor (SCF), a granulocyte colony-stimulating factor (G-CSF) [J. Biol. Chem., 258, 9017 (1983)], a macrophage colony-stimulating factor (M-CSF) [J. Exp. Med., 173, 269 (1992)], a granulocyte-macrophage colony-stimulating factor (GM-CSF) [J. Biol. Chem., 252, 1998 (1977)], interferon α, interferon β, interferon γ, interleukin-2 (IL-2) [Science, 193, 1007 (1976)], interleukin 6, interleukin 10, interleukin 11, interleukin-12 (IL-12) [J. Leuc. Biol., 55, 280 (1994)], a soluble interleukin 4 receptor, tumor necrosis factor α, DNase I, galactosidase, α-glucosidase, glucocerebrosidase, hemoglobin or transferrin, or derivatives thereof, and partial fragments of the glycoproteins.

The antibody may be any antibody having an antigen binding activity, and examples thereof include an antibody that recognizes a tumor-associated antigen or an antibody fragment thereof, an antibody that recognizes an antigen associated with allergy or inflammation or an antibody fragment thereof, an antibody that recognizes an antigen associated with a cardiovascular disease or an antibody fragment thereof, an antibody that recognizes an antigen associated with an autoimmune disease or an antibody fragment thereof, and an antibody that recognizes an antigen associated with a virus or a bacterial infection or an antibody fragment thereof.

Examples of the tumor-associated antigen include CD1a, CD2, CD3, CD4, CD5, CD6, CD7, CD9, CD10, CD13, CD19, CD20, CD21, CD22, CD25, CD28, CD30, CD32, CD33, CD38, CD40, CD40 ligand (CD40L), CD44, CD45, CD46, CD47, CD52, CD54, CD55, CD56, CD59, CD63, CD64, CD66b, CD69, CD70, CD74, CD80, CD89, CD95, CD98, CD105, CD134, CD137, CD138, CD147, CD158, CD160, CD162, CD164, CD200, CD227, adrenomedullin, angiopoietin related protein 4 (ARP4), aurora, B7-H1, B7-DC, integlin, bone marrow stromal antigen 2 (BST2), CA125, CA19.9, carbonic anhydrase 9 (CA9), cadherin, cc-chemokine receptor (CCR) 4, CCR7, a carcinoembryonic antigen (CEA), cysteine-rich fibroblast growth factor receptor-1 (CFR-1), c-Met, c-Myc, collagen, CTA, a connective tissue growth factor (CTGF), CTLA-4, cytokeratin-18, DF3, E-catherin, an epidermal growth factor receptor (EGFR), EGFRv III, EGFR2 (HER2), EGFR3 (HER3), EGFR4 (HER4), endoglin, an epithelial cell adhesion molecule (EpCAM), an endothelial protein C receptor (EPCR), ephrin, an ephrin receptor (Eph), EphA2, endotheliase-2 (ET2), FAM3D, a fibroblast activating protein (FAP), Fc receptor homolog 1 (FcRH1), ferritin, fibroblast growth factor 8 (FGF8), an FGF8 receptor, basic FGF (bFGF), a bFGF receptor, FGF receptor (FGFR) 3, FGFR4, FLT1, FLT3, a folate receptor, frizzled homologue 10 (FZD10), frizzled receptor 4 (FZD-4), G250, a G-CSF receptor, ganglioside (e.g., GD2, GD3, GM2, or GM3), globo H, gp75, gp88, GPR-9-6, heparanase 1, a hepatocyte growth factor (HGF), an HGF receptor, an HLA antigen (e.g., HLA-DR), HM1.24, a human milk fat globe (HMFG), hRS7, heat shock protein 90 (hsp90), idiotype epitope, an insulin-like growth factor (IGF), an IGF receptor (IGFR), interleukin (e.g., IL-6 or IL-15), an interleukin receptor (e.g., IL-6R or IL-15R), integrin, immune receptor translocation associated-4 (IRTA-4), kallikrein 1, KDR, KIR2DL1, KIR2DL2/3, KS1/4, lamp-1, lamp-2, laminin-5, Lewis y, sialyl Lewis x, a lymphotoxin-beta receptor (LTBR), LUNX, melanoma-associated chondroitin sulfate proteoglycan (MCSP), mesothelin, MICA, a Mullerian inhibiting substance type II receptor (MISIIR), mucin, a neural cell adhesion molecule (NCAM), Necl-5, Notch1, osteopontin, a platelet-derived growth factor (PDGF), a PDGF receptor, platelet factor-4 (PF-4), a phosphatidylserine, a prostate specific antigen (PSA), an prostate stem cell antigen (PSCA), a prostate specific membrane antigen (PSMA), a parathyroid hormone related protein/peptide (PTHrP), a receptor activator of NF-kappaB ligand (RANKL), a receptor for hyaluronic acid mediated motility (RHAMM), ROBO1, SART3, semaphorin 4B (SEMA4B), a secretory Leukocyte protease inhibitor (SLPI), SM5-1, sphingosine-1-phosphate, tumor-associated glycoprotein-72 (TAG-72), a transferrin receptor (TfR), TGF-beta, Thy-1, Tie-1, a Tie2 receptor, T cell immunoglobulin domain and mucin domain 1 (TIM-1), a human tissue factor (hTF), a Tn antigen, a tumor necrosis factor (TNF), a Thomsen-Friedenreich antigen (a TF antigen), a TNF receptor, a tumor necrosis factor-related apoptosis-inducing ligand (TRAIL), a TRAIL receptor (e.g., DR4 or DR5), a system ASC amino acid transporter 2 (ASCT2), trkC, TROP-2, TWEAK receptor Fn14, a type IV collagenase, a urokinase receptor, a vascular endothelial growth factor (VEGF), a VEGF receptor (e.g., VEGFR1, VEGFR2, or VEGFR3), vimentin, and VLA-4.

The antibody may be either a monoclonal antibody or a polyclonal antibody. Examples of a class of the antibody include immunoglobulin G (IgG), immunoglobulin A (IgA), immunoglobulin E (IgE), and immunoglobulin M (IgM), and IgG is preferred. Examples of a subclass of IgG include IgG1, IgG2, IgG3, and IgG4.

The antibody includes, for example, a fragment containing a part of an antibody, and examples thereof include a Fragment of antigen binding (Fab), Fab′, F(ab′)2, a single chain antibody (single chain Fv, scFv) and a disulfide stabilized antibody (disulfide stabilized Fv, dsFv), and a fusion protein containing an Fc region of an antibody.

Examples of the antibody include an antibody secreted by hybridoma cells prepared from spleen cells of animals immunized with an antigen, and an antibody produced by a gene recombination technique, i.e., an antibody obtained by introducing, into host cells, an antibody expression vector to which an antibody gene is inserted. Specific examples thereof include an antibody produced by hybridomas, a human chimeric antibody, a humanized antibody, and a human antibody.

The human chimeric antibody means an antibody composed of an antibody heavy chain variable region (hereinafter also referred to as HV or VH as the heavy chain as an H chain and the variable region as a V region) and an antibody light chain variable region (hereinafter also referred to as LV or VL as the light chain as an L chain) of a non-human animal, and a human antibody heavy chain constant region (hereinafter also referred to as CH as the constant region as a C region) and a human antibody light chain constant region (hereinafter also referred to as CL). As the non-human animal, any animals such as mice, rats, hamsters, and rabbits can be used as long as hybridomas can be prepared.

The human chimeric antibody can be produced by obtaining a cDNA encoding the VH and VL from hybridomas for producing a monoclonal antibody, inserting the cDNA into a host cell expression vector having a gene encoding the human antibody CH and the human antibody CL, constructing a human chimeric antibody expression vector, and introducing the human chimeric antibody expression vector into a host cell.

The CH of the human chimeric antibody may be any one belonging to human immunoglobulin (hereinafter referred to as hIg), and is preferably of an hIgG class. Further, any of subclasses such as hIgG1, hIgG2, hIgG3, or hIgG4 belonging to the hIgG class can be used. The CL of the human chimeric antibody may be any one belonging to hIg, and those of a κ class or a λ class can be used.

Examples of the humanized antibody include a CDR-grafted antibody prepared by grafting an amino acid sequence of a human complementarity determining region (hereinafter referred to as CDR) of the antibody VH and VL of the non-human animal to an appropriate position of the human antibody VH and VL.

The CDR-grafted antibody can be produced by constructing a cDNA encoding a V region which is obtained by grafting a CDR sequence of the antibody VH and VL of the non-human animal into a CDR sequence of any human antibody VH and VL, inserting the cDNA into a host cell expression vector having a gene encoding the human antibody CH and the human antibody CL to construct a CDR-grafted antibody expression vector, and introducing the expression vector into a host cell to express the CDR-grafted antibody.

The CH of the CDR-grafted antibody may be any one belonging to hIg, and is preferably of an hIgG class. Further, any of subclasses such as hIgG1, hIgG2, hIgG3, or hIgG4 belonging to the hIgG class can be used. The CL of the CDR-grafted antibody may be any one belonging to hIg, and those of a κ class or a λ class can be used.

The human antibody can be obtained, for example, by isolating human peripheral lymphocytes, immortalizing the human peripheral lymphocytes by infection with EB virus or the like, cloning the human peripheral lymphocytes, and culturing the lymphocytes that produce the antibody, and purifying the antibody from a culture.

The human antibody can be prepared from a human antibody phage library. The human antibody phage library is a library in which an antibody fragment such as Fab or scFv is expressed on a phage surface by inserting an antibody gene prepared from a human B cell into a phage gene. From the library, a phage expressing an antibody fragment having an antigen binding activity can be collected by using the binding activity to an immobilized antigen as an indicator. The antibody fragment can be converted into a human antibody molecule consisting of two complete H chains and two complete L chains.

The human antibody can also be produced by obtaining a cDNA encoding the VL and VH from a human antibody-producing hybridoma, inserting the cDNA into an animal cell expression vector having a DNA encoding the human antibody CL and CH, in which one or more amino acid residues of a wild-type (hereinafter, referred to as WT) are substituted with Cys residues as appropriate by the above-described method or the like, and introducing the expression vector into an animal cell.

The human antibody-producing hybridoma can be obtained from a human antibody-producing transgenic animal by a hybridoma producing method commonly practiced for mammals other than humans. The human antibody-producing transgenic animal refers to an animal with a human antibody gene incorporated into a cell thereof. Specifically, a human antibody-producing transgenic mouse can be prepared by introducing a human antibody gene into a mouse ES cell and grafting the ES cell into a mouse initial embryo [Proc. Natl. Acad. Sci. USA, 97, 722 (2000)].

Alternatively, the human antibody can also be produced by obtaining a cDNA encoding the VL and VH from a human antibody-producing hybridoma, inserting the cDNA into an animal cell expression vector having a DNA encoding the human antibody CL and CH, further substituting one or more amino acid residues of WT with Cys residues as appropriate by the above-described method or the like to construct a human antibody expression vector, and introducing the human antibody expression vector into an animal cell for expression.

The CH of the WT used for the human antibody may be any one belonging to hIg, and is preferably of an hIgG class. Further, any of subclasses such as hIgG1, hIgG2, hIgG3, and hIgG4 belonging to the hIgG class can be used. The CL of the human antibody may be any one belonging to hIg, and those of a k class or a λ class can be used.

Specific examples of the antibody produced by the method of the present invention include, but are not limited to, the following antibodies.

Examples of the antibody that recognizes the tumor-associated antigen include an anti-GD2 antibody [Anticancer Res., 13, 331 (1993)], an anti-GD3 antibody [Cancer Immunol. Immunother., 36, 260 (1993)], an anti-GM2 antibody [Cancer Res., 54, 1511 (1994)], an anti-HER2 antibody [Proc. Natl. Acad. Sci. USA, 89, 4285 (1992), U.S. Pat. No. 5,725,856], an anti-CD52 antibody [Proc. Natl. Acad. Sci. USA, 89, 4285 (1992)], an anti-MAGE antibody [British J. Cancer, 83, 493 (2000)], an anti-HM1.24 antibody [Molecular Immunol., 36, 387 (1999)], an anti-parathyroid hormone-related protein (PTHrP) antibody [Cancer, 88, 2909 (2000)], an anti-bFGF antibody, an anti-FGF-8 antibody [Proc. Natl. Acad. Sci. USA, 86, 9911 (1989)], an anti-bFGFR antibody, an anti-FGF-8R antibody [J. Biol. Chem., 265, 16455 (1990)], an anti-IGF antibody [J. Neurosci. Res., 40, 647 (1995)], an anti-IGF-IR antibody [J. Neurosci. Res., 40, 647 (1995)], an anti-PSMA antibody [J. Urology, 160, 2396 (1998)], an anti-VEGF antibody [Cancer Res., 57, 4593 (1997)], an anti-VEGFR antibody [Oncogene, 19, 2138 (2000), WO 96/30046], an anti-CD20 antibody [Curr. Opin. Oncol., 10, 548 (1998), U.S. Pat. No. 5,736,137 specification], an anti-CD10 antibody, an anti-EGFR antibody (WO 96/402010), an anti-Apo-2R antibody (WO 98/51793), an anti-ASCT2 antibody (WO 2010/008075), an anti-CEA antibody [Cancer Res., 55 (23 suppl): 5935s-5945s, (1995)], an anti-CD38 antibody, an anti-CD33 antibody, an anti-CD22 antibody, an anti-EpCAM antibody, and an anti-A33 antibody.

Examples of the antibody that recognizes an antigen associated with allergy or inflammation include an anti-interleukin 6 antibody [Immunol. Rev., 127, 5 (1992)], an anti-interleukin 6 receptor antibody [Molecular Immunol., 31, 371 (1994)], an anti-interleukin 5 antibody [Immunol. Rev., 127, 5 (1992)], an anti-interleukin 5 receptor antibody, an anti-interleukin 4 antibody [Cytokine, 3, 562 (1991)], an anti-interleukin 4 receptor antibody [J. Immunol. Methods, 217, 41 (1998)], an anti-tumor necrosis factor antibody [Hybridoma, 13, 183 (1994)], an anti-tumor necrosis factor receptor antibody [Molecular Pharmacol., 58, 237 (2000)], an anti-CCR4 antibody [Nature, 400, 776, (1999)], an anti-chemokine antibody (Peri et al., J. Immunol. Meth., 174, 249, 1994), and an anti-chemokine receptor antibody [J. Exp. Med., 186, 1373 (1997)].

Examples of the antibody that recognizes an antigen associated with a cardiovascular disease include an anti-GPIIb/IIIa antibody [J. Immunol., 152, 2968 (1994)], an anti-platelet-derived growth factor antibody [Science, 253, 1129 (1991)], an anti-platelet-derived growth factor receptor antibody [J. Biol. Chem., 272, 17400 (1997)], an anti-blood coagulation factor antibody [Circulation, 101, 1158 (2000)], an anti-IgE antibody, an anti-αVβ3 antibody, and an α4β7 antibody.

Examples of the antibody that recognizes an antigen associated with a virus or a bacterial infection include an anti-gp120 antibody [Structure, 8, 385 (2000)], an anti-CD4 antibody [J. Rheumatology, 25, 2065 (1998)], an anti-CCR5 antibody, and an anti-verotoxin antibody [J. Clin. Microbiol., 37, 396 (1999)].

Producing the target protein at a high concentration means producing the target protein such that a target protein concentration in the culture medium at the end of the cell culture is, for example, 1.5 times or more, more preferably 2 times or more, and even more preferably 3 times or more as compared with culture using normal cells. Specifically, for example, the target protein concentration in the culture medium at the end of the cell culture is preferably 2 g/L or more, more preferably 3 g/L or more, even more preferably 4 g/L or more, and particularly preferably 5 g/L or more. An upper limit of the target protein concentration in the culture medium at the end of the cell culture is not particularly limited, and the target protein concentration is typically preferably 6 g/L or less.

When the target protein is an antibody, an antibody concentration in the culture medium at the end of the cell culture is preferably 4.0 g/L or more, more preferably 5.0 g/L or more, and even more preferably 6.0 g/L or more. An upper limit of the antibody concentration in the culture medium at the end of the cell culture is not particularly limited, and the antibody concentration is preferably 8.0 g/L or less.

### <Medium>

In the present invention, examples of the medium used for cell culture include a powder medium, a liquid medium, and a slurry medium. The medium can be appropriately selected from commercially available media, and two or more types of media may be mixed. Further, known media and the like described in the literature can also be selected.

Examples of the medium include a bacterium cell culture medium, a yeast cell culture medium, a plant cell culture medium, and an animal cell culture medium. Among them, an animal cell culture medium is preferred. The medium is not particularly limited, and examples thereof include an expansion culture medium, a basal (initial) medium, and a feed medium.

The medium may be any of a synthetic medium, a semi-synthetic medium, and a natural medium. Examples thereof include a basal medium, a serum-containing medium, a serum-free medium, an animal-derived component-free medium, and a protein-free medium. Among them, a serum-free medium, a protein-free medium, or a completely synthetic medium is preferred.

As the cell culture medium, an animal cell culture medium is preferred, and a Chinese hamster ovarian tissue-derived CHO cell culture medium is more preferred.

Examples of the basal medium include commercially available media such as an RPMI1640 medium [The Journal of the American Medical Association, 199, 519 (1967)], an Eagle's MEM medium [Science, 122, 501 (1952)], a Dulbecco's modified MEM (DMEM) medium [Virology, 8, 396 (1959)], a 199 medium [Proceeding of the Society for the Biological Medicine, 73, 1 (1950)], an F12 medium (manufactured by LTI) [Proc. Natl. Acad. Sci. USA, 53, 288 (1965)], an Iscove's modified Dulbecco's medium (an IMDM medium) [J. Experimental Medicine, 147, 923 (1978)], an EX-CELL (registered trademark) 302 medium and an EX-CELL (registered trademark) 325 medium (manufactured by SAFC Biosciences), and a CHO-S-SFMII medium (manufactured by Invitrogen), or modified or mixed media thereof. Among them, an RPMI 1640 medium, a DMEM medium, an F12 medium, IMDM, an EX-CELL (registered trademark) 302 medium, or a hybridoma SFM medium (manufactured by Invitrogen) is preferred.

Examples of the serum-containing medium include a basal medium supplemented with one or more kinds of serum or serum fractions from serum of mammal animals such as bovine or horse, serum of bird animals such as chicken, serum of fish animals such as yellowtail, or fractions of the serum.

Examples of the serum-free medium include a basal medium supplemented with a serum substitute such as a nutritional factor or a biologically active substance.

In the animal-derived component-free medium, a substance added instead of the animal-derived component may be added. Examples of the substance include a biologically active substance produced by a gene recombination method, a hydrolysate or an animal-derived raw material-free lipid.

Examples of the protein-free medium include an animal derived protein free medium (an ADPF medium, manufactured by Hyclone), a CD-hybridoma medium (manufactured by Invitrogen), a CD-CHO medium (manufactured by Invitrogen), an IS-CD-CHO medium (manufactured by Irvine Scientific), or an EX-CELL (registered trademark) CD-CHO medium (manufactured by SAFC Biosciences).

A method for producing the powder medium is not particularly limited, and preferred examples thereof include a production method by a mixing process such as disk milling, ball milling, or pin milling of dry components, or a production method by freeze-drying a pre-made aqueous solution.

The powder medium includes a medium present in granular form.

A method for producing the powder medium present in granular form is not particularly limited, and examples thereof include an advanced granulation technology (registered trademark). A step of further spraying a solution obtained by dissolving at least one material selected from the group consisting of natural glue, synthetic glue, saccharides, and fats and oils to a finely granulated component, followed by drying, may be included.

A desired nutritional factor may be appropriately selected and added to the medium. Further, the medium may be composed of components appropriately selected for the desired nutritional factor. Examples of the nutritional factor include a carbon source such as saccharides and a nitrogen source such as an amino acid. Specific examples thereof include an amino acid, a metal, a vitamin, saccharides, a salt, a lipid, a nucleic acid, a biologically active substance, a fatty acid, an organic acid, a protein, and a hydrolysate. The compounds may form a salt such as a hydrochloride, a sodium salt, a potassium salt, and an ammonium salt, and/or a solvate such as a hydrate.

The amino acid is not particularly limited, and examples thereof include L-alanine (Ala), L-arginine (Arg), L-asparagine (Asn), L-aspartic acid (Asp), L-cysteine (Cys), L-cystine, L-glutamic acid (Glu), L-glutamine (Gln), glycine (Gly), L-histidine (His), L-isoleucine (Ile), L-leucine (Leu), L-lysine (Lys), L-methionine (Met), L-phenylalanine (Phe), L-proline (Pro), L-serine (Ser), L-threonine (Thr), L-tryptophan (Trp), and L-valine (Val). The amino acid may be used alone or in combination of two or more kinds thereof. A salt such as a hydrochloride or a sodium salt and/or a solvate such as a hydrate thereof may be used. The amino acid may be added as a peptide, and examples thereof include L-alanyl-L-glutamine and L-alanyl-L-cysteine.

Examples of the biologically active substance include insulin, transferrin, serum albumin, and a growth factor-containing serum fraction.

Examples of the lipid include cholesterol, linoleic acid, and linolenic acid. A salt such as a hydrochloride or a sodium salt and/or a solvate such as a hydrate thereof may be used.

The metal is not particularly limited, and examples thereof include iron, manganese, zinc, molybdenum, vanadium, copper, cadmium, rubidium, cobalt, zirconium, germanium, nickel, tin, chromium, and silicon. The metal may be used alone or in combination of two or more kinds thereof. The metal may form, for example, a salt such as a hydrochloride, a sulfate, a sodium salt, a potassium salt or an ammonium salt, and/or a solvate such as a hydrate.

The saccharides may be a monosaccharide, an oligosaccharide or a polysaccharide, and are not particularly limited. Further, the saccharides also include a sugar derivative such as a deoxy sugar, a uronic acid, an amino sugar, or a sugar alcohol. Examples thereof include glucose, mannose, galactose, fructose, ribose, arabinose, ribulose, erythrose, erythrulose, glyceraldehyde, dihydroxyacetone, sedoheptulose, maltose, lactose, and sucrose. The saccharides may be used alone or in combination of two or more kinds thereof. A salt such as a hydrochloride or a sodium salt and/or a solvate such as a hydrate thereof may be used.

The vitamin is not particularly limited, and examples thereof include d-biotin, D-pantothenic acid, choline, folic acid, myo-inositol, niacinamide, pyridoxl, riboflavin, thiamine, cyanocobalamin, and DL-α-tocopherol. The vitamin may be used alone or in combination of two or more kinds thereof. A salt such as a hydrochloride or a sodium salt and/or a solvate such as a hydrate thereof may be used.

Examples of the hydrolysate include a hydrolysate or an extract of a soybean, wheat, rice, peas, cottonseed, fish or a yeast extract. Specific example thereof include SOY HYDROLYSATE UF (Catalog No.: 91052-1K3986 or 91052-5K3986, manufactured by SAFC Bioscience).

### <Cell>

The cell may be either a eukaryotic cell or a prokaryotic cell, and examples thereof include a cell derived from mammals, birds, reptiles, amphibians, fishes, insects, or plants, microorganisms such as a bacterium, an Escherichia coli, or a Bacillus subtilis, a cell derived from microorganisms such as a bacterium, an Escherichia coli, or a Bacillus subtilis, a yeast, or a cell derived from a yeast or the like.

Among them, an animal cell belonging to mammals is preferred, an animal cell derived from primates such as humans and monkeys, or an animal cell derived from rodents such as mice, rats, or hamsters is more preferred, and a Chinese hamster ovary tissue-derived CHO cell is most preferred.

The Chinese hamster ovary tissue-derived CHO cell in the present invention includes any cell as long as it is a cell established from Chinese hamster (Cricetulus griseus) ovary tissue.

Specific examples thereof include a CHO cell described in the literature, such as Journal of Experimental Medicine, 108, 945 (1958), Proc. Natl. Acad. Sci. USA, 60, 1275 (1968), Genetics, 55, 513 (1968), Chromosoma, 41, 129 (1973), Methods in Cell Science, 18, 115 (1996), Radiation Research, 148, 260 (1997), Proc. Natl. Acad. Sci. USA, 77, 4216 (1980), Proc. Natl. Acad. Sci. 60, 1275 (1968), Cell, 6, 121 (1975), and Molecular Cellgenetics, Appendix I, II, 883-900.

Examples thereof also include a CHO-K1 strain (ATCC No. CCL-61), a DUXB11 strain (ATCC CRL-9096), a Pro-5 strain (ATCC CRL-1781), and a CHO/dhfr-(ATCC No. CRL-9096), which are registered in The American Type Culture Collection (ATCC), a commercially available CHO-S strain (Cat #11619, manufactured by Life technologies) or CHO/DG44 [Proc. Natl. Acad. Sci. USA, 77, 4216 (1980)], or substrains obtained by adapting the strains to various media.

Examples of the cell belonging to mammals include a myeloma cell, an ovarian cell, a kidney cell, a blood cell, a uterine cell connective tissue cell, a mammary gland cell, an embryonic retina blast cell, and cells derived from these cells. Among them, a myeloma cell, a cell derived from a myeloma cell, an ovarian cell, or a cell derived from an ovarian cell is preferred.

Examples thereof include a human cell strain such as HL-60 (ATCC No. CCL-240), HT-1080 (ATCC No. CCL-121), HeLa (ATCC No. CCL-2), 293 (ECACC No. 85120602), Namalwa (ATCC CRL-1432), Namalwa KJM-1 [Cytotechnology, 1, 151 (1988)], NM-F9 (DSM ACC2605, WO 2005/017130) and PER. C6 (ECACC No. 96022940, U.S. Pat. No. 6,855,544 specification), a monkey cell strain such as VERO (ATCC No. CCL-1651) and COS-7 (ATCC No. CRL-1651), a mouse cell strain C127I (ATCC No. CRL-1616), Sp2/0-Ag14 (ATCC No. CRL-1581), NIH3T3 (ATCC No. CRL-1658), and NS0 (ATCC No. CRL-1827), a rat cell strain such as Y3 Ag1.2.3. (ATCC No. CRL-1631), YO (ECACC No. 85110501), and YB2/0 (ATCC No. CRL-1662), a hamster cell strain such as the Chinese hamster ovary tissue-derived CHO cells described above and BHK21 (ATCC No. CRL-10), and a canine cell such as MDCK (ATCC No. CCL-34).

Examples of the cell belonging to birds include a chicken cell strain SL-29 (ATCC No. CRL-29). Examples of the cell belonging to fishes include a zebrafish cell strain ZF4 (ATCC No. CRL-2050).

Examples of the cell belonging to insects include a moth (Spodoptera frugiperda) cell strain Sf9 (ATCC No. CRL-1711). Examples of a primary cultured cell used for vaccine production include a primary monkey kidney cell, a primary rabbit kidney cell, a primary chicken fetal cell, and a primary quail fetal cell.

Examples of the myeloma cell or the cell derived from a myeloma cell include Sp2/0-Ag14, NS0, Y3 Ag1.2.3., YO, and YB2/0. Examples of the ovarian cell or the cell derived from an ovarian cell include the Chinese hamster ovarian tissue-derived CHO cell described above. Examples of the kidney cell include 293, VERO, COS-7, BHK21, and MDCK.

Examples of the blood cell include HL-60, Namalwa, Namalwa KJM-1, and NM-F9. Examples of the uterine cell include HeLa. Examples of the connective tissue cell include HT-1080 and NIH 3T3. Examples of the mammary gland cell include C1271I. Examples of the embryonic retina blast cell include PER.C6.

The cell is not particularly limited in terms of whether it has the ability to produce the target protein, and examples thereof include an iPS cell obtained by introducing several types of genes into a somatic cell, a sperm or an oocyte obtained from a mammalian donor including humans, a target protein-producing cell, and a target protein-producing fusion cell. Among them, a target protein-producing cell or a target protein-producing fusion cell is preferred, and a target protein-producing animal cell or a target protein-producing animal-derived fusion cell is more preferred. For example, when the target protein is an antibody, examples of the cell include a hybridoma which is a fusion cell of a myeloma cell and an antibody-producing cell such as a B cell. The animal cell also includes an animal cell that is mutated to produce the target protein, or an animal cell that is mutated to increase an expression level of the target protein.

Examples of the animal cell that is mutated to produce the target protein include a cell in which a protein modifying enzyme is mutated or the like so as to produce the target protein. For example, when the target protein is a glycoprotein, examples thereof include a cell in which various sugar chain modifying enzymes are mutated so as to change a sugar chain structure.

Further, as the target protein-producing animal cell, any animal cell may be used as long as the target protein can be produced, and for example, the target protein-producing animal cell also includes an animal cell transformed with a recombinant vector containing a gene involved in production of the target protein. The transformed cell can be obtained by introducing a DNA involved in production of the target protein and a recombinant vector containing a promoter into the cell belonging to the mammal.

As the gene involved in the production of the target protein, for example, any of a DNA encoding the target protein, a DNA encoding an enzyme or protein involved in biosynthesis of the target protein, and the like can be used.

As the promoter, any promoter can be used as long as it functions in the animal cell used in the present invention, and examples thereof include a promoter of immediate early (IE) gene of cytomegalovirus (CMV), an SV40 early promoter, a retroviral promoter, a metallothionein promoter, a heat shock promoter, and an SRa promoter. A human CMV IE gene enhancer or the like may be used together with the promoter.

The recombinant vector can be prepared using a desired vector. As the vector used for preparing the recombinant vector, any vector can be used as long as it functions in the animal cell used in the present invention, and examples thereof include pcDNAI, pcDM8 (manufactured by Funakoshi Co., Ltd.), pAG107 [JP 3-22979 A, Cytotechnology, 3, 133 (1990)], pAS3-3 (JP 2-227075 A), pcDM8 [Nature, 329, 840 (1987)], pcDNAI/Amp (manufactured by Invitrogen), pREP4 (manufactured by Invitrogen), pAG103 [J. Biochem., 101, 1307 (1987)], and pAG210.

As a method for introducing a recombinant vector into a host cell, any method of introducing a DNA into the cell can be used, and examples thereof include an electroporation method [Cytotechnology, 3, 133 (1990)], a calcium phosphate method (JP 2-227075 A) or a lipofection method [Proc. Natl. Acad. Sci. USA, 84, 7413 (1987), Virology, 52, 456 (1973)].

Specific examples of the transformed cell include an anti-GD3 human chimeric antibody-producing transformed cell 7-9-51 (FERM BP-6691), an anti-CCR4 chimeric antibody-producing transformed cell KM2760 (FERM BP-7054), an anti-CCR4 humanized antibody-producing transformed cells KM8759 (FERM BP-8129), KM8760 (FERM BP-8130), and 709LCA-500D (FERM BP-8239), an anti-IL-5 receptor α chain chimeric antibody-producing transformed cell KM7399 (FERM BP-5649), anti-IL-5 receptor α-chain human CDR-grafted antibody-producing transformed cells KM8399 (FERM BP-5648) and KM9399 (FERM BP-5647), anti-GM2 human CDR-grafted antibody-producing transformed cells KM8966 (FERM BP-5105), KM8967 (FERM BP-5106), KM8969 (FERM BP-5527), and KM8970 (FERM BP-5528), an anti-CD20 antibody-producing transformant Ms704-CD20 (FERM BP-10092), and an antithrombin III-producing transformed cell Ms705-pKAN-ATIII (FERM BP-8472).

### <LMWS>

The LMWS is a degradation product of the target protein. A generation amount of the LMWS can be measured by subjecting the culture medium to affinity purification and then performing capillary electrophoresis under non-reduction conditions. In the present description, the generation amount (%) of the LMWS (hereinafter, also abbreviated as an “LMWS amount”) refers to a value obtained by cutting peaks from a chart obtained by the capillary electrophoresis and dividing an LMWS peak area by a total peak area. The generation amount of the LMWS is preferably measured at the end of the cell culture, specifically, for example, 13 days after the start of culture.

In the method of the present invention, it is preferred that the generation amount of the LMWS is reduced as compared with a cell culture process not including the means for removing the reactive oxygen species in the culture medium. Specifically, the generation amount of the LMWS is preferably reduced by 0.1% or more, more preferably reduced by 0.5% or more, and even more preferably reduced by 1.0% or more as compared with the cell culture process not including the means for removing the reactive oxygen species in the culture medium.

### <Means for Removing Reactive Oxygen Species>

The method of the present invention includes the means for removing the reactive oxygen species in the culture medium. The means for removing the reactive oxygen species in the culture medium is preferably at least one selected from the following (a) to (e).
(a) Adding an antioxidant to a medium used for the cell culture
(b) Making a cystine or cystine analogue concentration in the culture medium at the end of the cell culture 1.90 mmol/L or less
(c) Making a copper concentration in the culture medium at the end of the cell culture 20.0 μmol/L or less
(d) Adding a chelating compound to a medium used for the cell culture
(e) Making a pH of a feed medium used for the cell culture 8.0 or more

Hereinafter, each means will be described.

### (a) Adding an Antioxidant to a Medium Used for the Cell Culture

Examples of the antioxidant include a catechin analogue, ascorbic acid, α-tocopherol, vitamin K, retinol, thiamine, riboflavin, glutathione, carotenoids, polyphenols, flavonoids, mannitol, taurine, N-acetylcysteine, uric acid, bilirubin, butylated hydroxyanisole, butylated hydroxytoluene, and tert-butylhydroquinone, and a catechin analogue is preferred. These may be used alone or in combination of two or more kinds thereof.

Examples of the catechin analogue include catechin hydrate, epicatechin, gallocatechin gallate, and epigallocatechin gallate, and catechin hydrate and epigallocatechin gallate are preferred.

Examples of the carotenoids include β-carotene, lutein, astaxanthin, and lycopene.

Examples of the polyphenols include quercetin, chlorogenic acid, and curcumin.

Examples of the flavonoids include an anthocyanin, a flavan, rutin, and an isoflavonoid.

By adding the antioxidant to the medium used for the cell culture, the radical chain reaction caused by the reactive oxygen can be prevented, and the LMWS amount can be reduced while the protein production amount can be maintained at a high level. A concentration of the antioxidant to be added to the medium can be appropriately adjusted depending on the type of the antioxidant, the target protein, or the cell to be used.

Specifically, for example, when the antioxidant is a catechin analogue, the antioxidant concentration in the culture medium at the end of the culture is preferably 50 μmol/L or more, more preferably 100 μmol/L or more, and even more preferably 190 μmol/L or more.

More specifically, for example, when the catechin analogue is epigallocatechin gallate, an epigallocatechin gallate concentration in the culture medium at the end of the culture is preferably 50 μmol/L to 300 μmol/L, more preferably 50 μmol/L to 250 μmol/L, and even more preferably 70 μmol/L to 200 μmol/L.

Even more specifically, for example, when the catechin analogue is catechin hydrate, a catechin hydrate concentration in the culture medium at the end of the culture is preferably 50 μmol/L to 450 μmol/L, more preferably 100 μmol/L to 400 μmol/L, and even more preferably 120 μmol/L to 350 μmol/L.

Specific examples of a method of making the antioxidant concentration in the culture medium at the end of the cell culture fall within the above range include the following method. A correlation between the antioxidant concentration in the culture medium at the start of the cell culture and the antioxidant concentration at the end of the cell culture is obtained in advance. Based on the correlation, the concentration of the antioxidant to be added to the medium at the start of the cell culture is set such that the antioxidant concentration in the culture medium at the end of the cell culture falls within the above range.

(b) Making a Cystine or Cystine Analogue Concentration in the Culture Medium at the End of the Cell Culture 1.90 mmol/L or Less

Examples of the cystine or cystine analogue include L-cystine, cystin dimethyl ester, cystin ethyl ester, cystine dihydrochloride, and an L-cystine disodium salt.

The cystine or cystine analogue concentration in the culture medium at the end of the cell culture is preferably 1.90 mmol/L or less, more preferably 1.60 mmol/L or less, and even more preferably 1.20 mmol/L or less. A lower limit of the cystine or cystine analogue concentration in the culture medium at the end of the cell culture is not particularly limited, and generally, the cystine or cystine analogue concentration is preferably 0.10 mmol/L or more, more preferably 0.20 mmol/L or more, even more preferably 0.50 mmol/L or more, and particularly preferably 1.00 mmol/L or more.

By making the cystine or cystine analogue concentration in the culture medium at the end of the cell culture 1.90 mmol/L or less, the radical chain reaction caused by the reactive oxygen can be prevented, and the LMWS amount can be reduced while the protein production amount can be maintained at a high level.

Specific examples of a method of making the cystine or cystine analogue concentration in the culture medium at the end of the cell culture fall within the above range include the following method. A correlation between the cystine or cystine analogue concentration in the culture medium at the start of the cell culture and the cystine or cystine analogue concentration at the end of the cell culture is obtained in advance. Based on the correlation, the concentration of the cystine or cystine analogue to be added to the medium at the start of the cell culture is set such that the cystine or cystine analogue concentration in the culture medium at the end of the cell culture falls within the above range.

(c) Making a Copper Concentration in the Culture Medium at the End of the Cell Culture 20.0 μmol/L or Less

The copper concentration in the culture medium at the end of the cell culture is preferably 20.0 μmol/L or less, more preferably 5 μmol/L or less, and even more preferably 0.50 μmol/L or less. A lower limit of the copper concentration in the culture medium at the end of the cell culture is not particularly limited, and the copper concentration is preferably 0.05 μmol/L or more, more preferably 0.10 μmol/L or more, and even more preferably 0.25 μmol/L or more.

By making the copper concentration in the culture medium at the end of the cell culture 20.0 μmol/L or less, the radical chain reaction caused by the reactive oxygen can be prevented, and the LMWS amount can be reduced while the protein production amount can be maintained at a high level.

Specific examples of a method of making the copper concentration in the culture medium at the end of the cell culture fall within the above range include the following method. A correlation between the copper concentration in the culture medium at the start of the cell culture and the copper concentration at the end of the cell culture is obtained in advance. Based on the correlation, the concentration of the copper to be added to the medium at the start of the cell culture is set such that the copper concentration in the culture medium at the end of the cell culture falls within the above range.

### (d) Adding a Chelating Compound to a Medium Used for the Cell Culture

Examples of the chelating compound include a group consisting of citric acid, ethylenediaminetetraacetic acid (EDTA), nitrilotriacetic acid (NTA), ethylenediamine-N,N′-disuccinic acid (EDDS), an oxalate, a tartrate, ethylene-bis(oxyethylenenitrilo)tetraacetic acid (EGTA), diethylenetriaminepentaacetic acid (DTPA), 5-sulfosalicylic acid, N,N-dimethyldodecylamine N-oxide, dithiooxamide, ethylenediamine, salicyaldoxime, N-(2′-hydroxyethyl)iminodiacetic acid (HIMDA), oxine quinolinol, and sulfoxine, and citric acid is preferred.

By adding the chelating compound to the medium used for the cell culture, the radical chain reaction caused by the reactive oxygen can be prevented, and the LMWS amount can be reduced while the protein production amount can be maintained at a high level. A concentration of the chelating compound to be added to the medium can be appropriately adjusted depending on the type of the chelating compound, the target protein, or the cell to be used.

Specifically, for example, when the chelating compound is citric acid, the chelating compound concentration in the culture medium at the end of the culture is preferably 1.50 μmol/L to 8.00 μmol/L, more preferably 1.80 μmol/L to 6.50 μmol/L, and even more preferably 1.80 μmol/L to 6.00 μmol/L.

Specific examples of a method of making the chelating compound concentration in the culture medium at the end of the cell culture fall within the above range include the following method. A correlation between the chelating compound concentration in the culture medium at the start of the cell culture and the chelating compound concentration at the end of the cell culture is obtained in advance. Based on the correlation, the concentration of the chelating compound to be added to the medium at the start of the cell culture is set such that the chelating compound concentration in the culture medium at the end of the cell culture falls within the above range.

### (e) Making a pH of a Feed Medium Used for the Cell Culture 8.0 or More

The feed medium means a medium to be added separately from the basal medium. The pH of the feed medium used for the cell culture is preferably 8.0 or more, more preferably 8.1 or more, and even more preferably 8.2 or more. An upper limit of the pH of the feed medium used for the cell culture is not particularly limited, and, for example, the pH of the feed medium is preferably 9.0 or less, more preferably 8.8 or less, and even more preferably 8.6 or less.

By making the pH of the feed medium used for the cell culture 8.0 or more, the radical chain reaction caused by the reactive oxygen can be prevented, and the LMWS amount can be reduced while the protein production amount can be maintained at a high level.

The pH of the feed medium can be adjusted using any acid or alkali. Specific examples of the acid or alkali include sodium hydrogen carbonate, hydrochloric acid, and sodium hydroxide.

The present invention also relates to a method for producing the target protein containing a reduced LMWS amount at a high concentration in a culture medium. The method includes a means for removing a reactive oxygen species in the culture medium. The expression “containing a reduced LMWS amount” means that an amount of the LMWS contained in the target protein is reduced as compared with a method not including the means for removing the reactive oxygen species in the culture medium. Specifically, for example, the amount of the LMWS contained in the target protein is preferably reduced by 0.1% or more, more preferably reduced by 0.5% or more, and even more preferably reduced by 1.0% or more, as compared with the method not including the means for removing the reactive oxygen species in the culture medium.

### <Method for Culturing Cells>

Examples of the method for culturing cells in the present invention include a method suitable for cells to be used, such as batch culture, repeat batch culture, rolling seed culture, fed-batch culture, or perfusion culture. Fed-batch culture is preferably used. Culture is generally performed under conditions of pH 6 to 8 and 30° C. to 40° C., for example, for 3 days to 20 days for fed-batch culture and for 3 days to 60 days for perfusion culture. If necessary during culture, an antibiotic such as streptomycin or penicillin may be added to the medium. Dissolved oxygen concentration control, pH control, temperature control, stirring, and the like can be performed by methods commonly used for culturing cells.

A culture volume in the culture method in the present invention may be a trace culture volume of generally 0.1 mL to 10 mL using a cell culture plate, a small culture volume of generally 10 mL to 1000 mL using an Erlenmeyer flask or the like, a large culture volume of generally 1 L to 20000 L using a culture tank such as a jar, which can be used for commercial production, or any culture volume.

### <Method for Purifying Protein>

The target protein produced by the method of the present invention can be isolated and purified using, for example, a general protein isolation and purification method or the like.

When the target protein is expressed in a dissolved state in cells, the cells are collected by centrifugation after the end of the culture, suspended in an aqueous buffer solution, and then disrupted with an ultrasonic disintegrator, a French press, a Manton-Gaurin homogenizer, a Dyno mill, or the like to obtain a cell-free extract.

From a supernatant obtained by centrifuging the cell-free extract, a crude or purified sample can be obtained by using a general protein isolation and purification method, that is, a solvent extraction method, a salting-out method with ammonium sulfate or the like, a desalting method, a precipitation method with an organic solvent, diethylaminoethyl-Sepharose, an anion exchange chromatography method using a resin such as DIAION HPA-75 (manufactured by Mitsubishi Chemical Corporation), a cation exchange chromatography method using a resin such as S-Sepharose FF (manufactured by Pharmacia), a hydrophobic chromatography method using a resin such as butyl sepharose and phenyl sepharose, a gel filtration method using molecular sieves, an affinity chromatography method using a resin containing protein A or protein G; a chromatofocusing method, and an electrophoresis method such as isoelectric focusing, alone or in combination.

When the target protein is extracellularly secreted, the protein can be collected in the culture supernatant. That is, a culture supernatant is obtained by processing the culture by a method such as centrifugation same as that described above, and a crude or purified sample can be obtained from the culture supernatant by using an isolation and purification method same as that described above.

### EXAMPLE

The present invention will be described in more detail with reference to the following Examples, but Examples are merely illustrative of the present invention and do not limit the scope of the present invention.

### [Example 1] Increase in LMWS Due to Application of Highly-Productive Process

With respect to an initial process, an effect of applying a highly-productive process on quality of a produced antibody was studied, and it was found that an LMWS tended to increase as productivity increased.

CHO cells introduced with an IgG expression gene (Mab A, Mab B, or Mab C) were seeded in a 2 L glass reactor or a 3 L SUS reactor containing a prepared animal cell production medium, and cultured for 13 days or 14 days. During a culture time, a feed medium was appropriately added. In the highly-productive process, a main raw material of the production medium and the feed medium, a culture time, a seeded viable cell density, a temperature, and a culture time are optimized with respect to the initial process using the productivity as an index (Table 1). An antibody concentration was measured using a Protein A HPLC.

TABLE 1

| | | | | Seeded | | | |
|---|---|---|---|---|---|---|---|
| | | | | viable cell | Dissolved | | |
| | | | | density | oxygen | | Culture |
| | | | | (×10⁵ | concentration | Temperature | time |
| Product | Isoform | Process | Medium | cells/mL) | (%) | (° C.) | (days) |
| MabA | IgG1 | Initial | Medium 1 | 3 | 50 | 37 | 13 |
| | | High-productive 1 | Medium 3 | 8 | 50 | 37 | 13 |
| | | High-productive 2 | Medium 4 | 8 | 50 | 37 | 13 |
| | | High-productive 3 | Medium 5 | 8 | 50 | 37 | 17 |
| MabB | IgG1 | Initial | Medium 1 | 3 | 50 | 37 | 13 |
| | | High-productive | Medium 2 | 20 | 50 | 37 | 14 |
| MabC | IgG4 | Initial | Medium 1 | 3 | 50 | 37 | 13 |
| | | High-productive | Medium 2 | 10 | 50 | 36 | 13 |

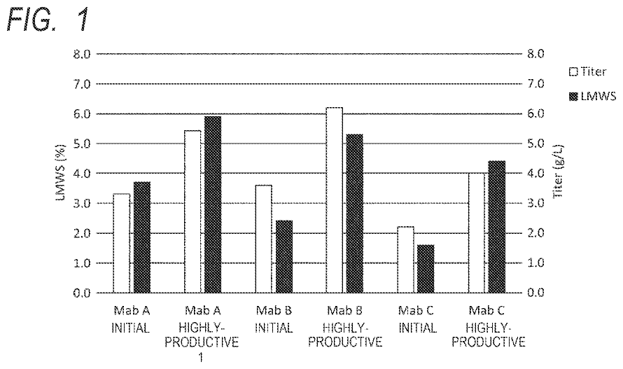

A produced antibody (Mab A, Mab B, or Mab C) was affinity-purified from a culture medium at the end of the culture using a Protein A resin, and subjected to capillary electrophoresis under non-reduction conditions using a Proteome Lab PA 800 plus (manufactured by AB Sciex) to evaluate an LMWS amount. The LMWS amount (%) was calculated by cutting peaks from a chart obtained by performing the capillary electrophoresis and dividing a peak area of the LMWS by a total peak area. The results are shown in FIG. 1.

As shown in FIG. 1, it was confirmed that, in a Mab A-producing strain, antibody productivity was 3.3 g/L in the initial process, but increased to 5.4 g/L when the highly-productive process 1 was applied. On the other hand, it was confirmed that the LMWS amount in the antibody was 3.7% in the initial process, but was 5.9% in the highly-productive process 1, and the LMWS amount in the antibody increased as the antibody production amount increased.

As shown in FIG. 1, it was confirmed that, in a Mab B-producing strain, antibody productivity was 3.6 g/L in the initial process, but increased to 6.2 g/L when the highly-productive process was applied. On the other hand, it was confirmed that the LMWS amount in the antibody was 2.4% in the initial process, but was 5.3% in the highly-productive process, and the LMWS amount in the antibody increased as the antibody production amount increased.

As shown in FIG. 1, it was confirmed that, in a Mab C-producing strain, antibody productivity was 2.2 g/L in the initial process, but increased to 4.0 g/L when the highly-productive process was applied. On the other hand, it was confirmed that the LMWS amount in the antibody was 1.6% in the initial process, but was 4.4% in the highly-productive process, and the LMWS amount in the antibody increased as the production amount of the antibody increased.

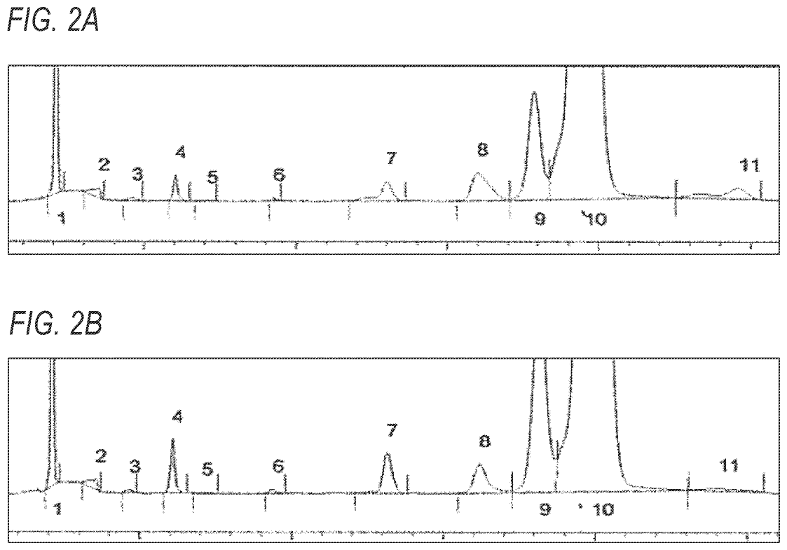

FIG. 2A shows an electropherogram for capillary electrophoresis of the Mab A in the initial process, FIG. 2B shows an electropherogram for capillary electrophoresis of the Mab A in the highly-productive process 1, and Table 2 shows an area % of an estimated molecular species for each peak. It was confirmed that, in the highly-productive process 1, in particular, HHL, HL, and L increased as the LMWS. Here, HHL represents a molecule obtained by deleting one L chain from a general antibody, HL represents a molecule having only one H chain and one L chain, and L represents an L chain molecule.

TABLE 2

| | | | | | LMWS |
|---|---|---|---|---|---|
| Estimated molecular species | L | HL | Fab-Fc | HHL | (Total) |
| Peak No. | 4 | 7 | 8 | 9 | NA |
| Initial process | 0.3 | 0.4 | 0.7 | 1.8 | 3.7 |
| High-productive process 1 | 0.5 | 0.6 | 0.5 | 3.5 | 5.9 |

Unit: %
NA: Not Applicable

From the above, it has become clear a new problem that the LMWS amount increased as the antibody productivity increased.

### [Example 2] LMWS Production Due to Hydrogen Peroxide Addition

For the purpose of elucidating a mechanism of LMWS production in a highly-productive process, a hydrogen peroxide addition test was performed as follows.

To Mab A (final concentration: 10 mg/mL) purified and buffer-substituted with PBS, hydrogen peroxide (Catalog No.: 081-04215, manufactured by FUJIFILM Wako Pure Chemical Corporation) or hydrogen peroxide and EDTA (Catalog No.: 06894-14, manufactured by Nacalai Tesque, Inc.) were added so as to satisfy the following conditions, followed by incubating at 37ºC for 17 days. After incubation, the buffer solution was substituted with PBS using an ultrafiltration membrane, and capillary electrophoresis was performed under non-reduction conditions using a Labchip GXII Touch HT (manufactured by PerkinElmer Co., Ltd.) to evaluate an LMWS amount.
Control: no additive
Condition 1: add hydrogen peroxide to a final concentration of 20 mmol/L
Condition 2: add hydrogen peroxide to a final concentration of 50 mmol/L
Condition 3: add hydrogen peroxide and EDTA to a final concentration of 20 mmol/L each

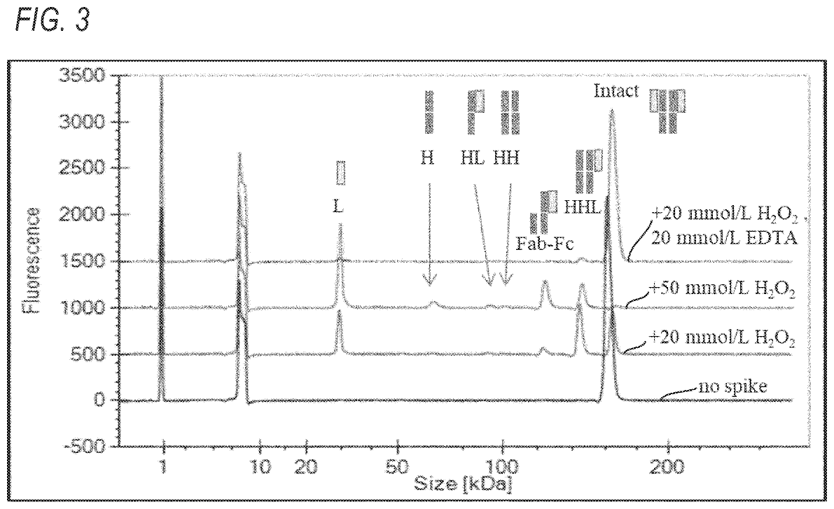

The results are shown in FIG. 3 and Table 3. Here, Intact represents a general antibody, Fab-Fc represents a molecule obtained by deleting one Fab from the general antibody, HH represents a molecule obtained by deleting two L chains from the general antibody, and H represents an H chain molecule.

TABLE 3

| Estimated molecular species | L | H | HL | HH | Fab-Fc | HHL | Intact |
|---|---|---|---|---|---|---|---|
| Control | 0.3 | ND | 0.1 | ND | 0.2 | 0.3 | 98.6 |
| Condition 1 | 33.8 | 0.9 | 1.4 | 0.6 | 4.4 | 31.6 | 26.1 |
| Condition 2 | 57.7 | 6.1 | 1.8 | 1.1 | 17.5 | 13.4 | 0.9 |
| Condition 3 | 1.8 | ND | 0.2 | ND | 0.3 | 1.5 | 95.2 |

Unit: %
ND: Not Detected

As shown in FIG. 3, it was confirmed that the antibody was degraded under a condition where hydrogen peroxide was added, and the LMWS including HHL, HL, and L increased. The LMWS production were prevented under a condition where EDTA having a chelating action was added in addition to hydrogen peroxide.

As described above, it has become clear that the molecular species of the LMWS generated in the highly-productive process in the culture process was common to the molecular species generated due to hydrogen peroxide addition. Hydrogen peroxide is a kind of reactive oxygen having oxidizing ability, and is known to produce hydroxyl radicals having even stronger oxidizing ability when reduced by divalent iron ions or the like. Since the LMWS production were prevented by adding EDTA to supplement divalent iron ions and the like, there was a possibility that the hydroxyl radicals among the reactive oxygen are directly related to the LMWS production. Therefore, it was suggested that the radical chain reaction caused by the reactive oxygen may be a factor in the mechanism of the LMWS production in the highly-productive process.

### [Example 3] LMWS Reduction Effect by Epigallocatechin Gallate Addition

An effect of adding epigallocatechin gallate having an antioxidant action to a medium used for cell culture on quality of an antibody produced in a highly-productive process was studied, and it was found that an LMWS was reduced.

A culture test was performed using a 250 mL baffled Erlenmeyer flask. Preparation of a medium and culture were performed by the following procedure. First, feed media with and without epigallocatechin gallate (Catalog No.: 02566-76, manufactured by Nacalai Tesque, Inc.) were prepared.

Next, CHO cells introduced with an IgG expression gene (Mab A or Mab C) were seeded in a 250 mL baffled Erlenmeyer flask containing the prepared production medium, and cultured with shaking in a CO₂ incubator for 13 days. The epigallocatechin gallate-containing feed medium was added such that an epigallocatechin gallate concentration in the culture medium at the end of the culture was 0 μmol/L, 77.7 μmol/L, or 193.7 μmol/L for Mab A and 0 μmol/L or 83.8 μmol/L for Mab C, respectively, or the epigallocatechin gallate-free feed medium was added, followed by performing fed-batch culture. Other various culture conditions thereof were highly-productive process 2 conditions for Mab A and highly-productive process conditions for Mab C (Table 1). An antibody concentration was measured using a Protein A HPLC.

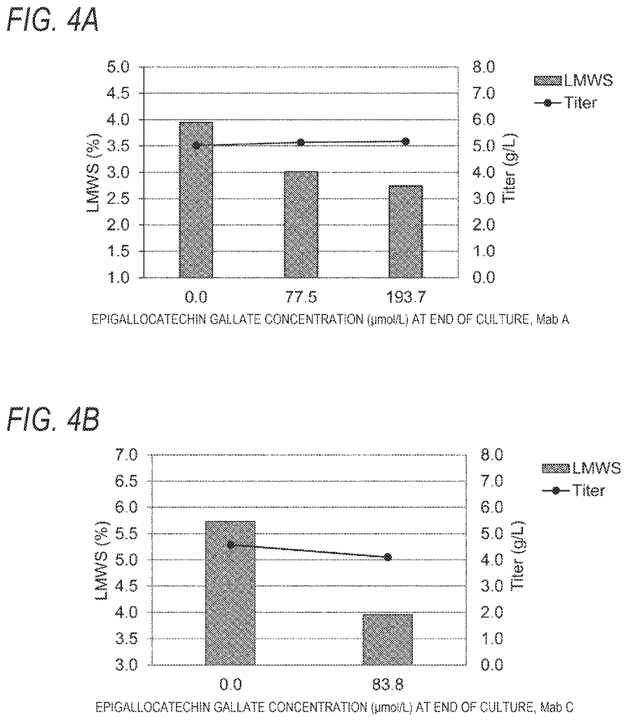

A produced antibody (Mab A or Mab C) was affinity-purified from the culture medium on day 13 of culture using a Protein A resin, and subjected to capillary electrophoresis under non-reduction conditions using a Labchip GXII touch HT (manufactured by PerkinElmer Co., Ltd.) to evaluate an LMWS amount. The results of the Mab A are shown in FIG. 4A, and the results of the Mab C are shown in FIG. 4B.

As shown in FIG. 4A, it was confirmed that the LMWS amount of the produced Mab A was 4.0% under a condition where the epigallocatechin gallate-free feed medium was added, was reduced to 3.0% under a condition where the epigallocatechin gallate concentration was 77.5 μmol/L, and was reduced to 2.7% under a condition where the epigallocatechin gallate concentration was 193.7 μmol/L.

As shown in FIG. 4A, an antibody concentration of the produced Mab A was 5.0 g/L under a condition where the epigallocatechin gallate-free feed medium was added, whereas the antibody concentration of the produced Mab A was 5.1 g/L under a condition where the epigallocatechin gallate concentration was 77.5 μmol/L, and was 5.2 g/L under a condition where the epigallocatechin gallate concentration was 193.7 μmol/L, and no reduction was observed.

As shown in FIG. 4B, it was confirmed that the LMWS amount of the produced Mab C was 5.7% under a condition where the epigallocatechin gallate-free feed medium was added, and was reduced to 4.0% under a condition where the epigallocatechin gallate concentration was 83.8 μmol/L.

As shown in FIG. 4B, it was confirmed that the antibody concentration of the produced Mab C was 4.6 g/L under a condition where the epigallocatechin gallate-free feed medium was added, whereas the antibody concentration of the produced Mab C was 4.1 g/L under a condition where the epigallocatechin gallate concentration was 83.8 μmol/L, which was equivalent.

From the above, it has become clear that the LMWS amount can be reduced while the antibody production amount can be maintained by adding epigallocatechin gallate into the medium under highly-productive process conditions.

### [Example 4] LMWS Reduction Effect by Catechin Hydrate Addition

An effect of adding catechin hydrate having an antioxidant action to a medium used for cell culture on quality of an antibody produced in a highly-productive process was studied, and it was found that an LMWS was reduced.

A culture test was performed using a 250 mL baffled Erlenmeyer flask. Preparation of a medium and culture were performed by the following procedure. First, feed media with and without catechin hydrate (Catalog No.: C0705, manufactured by Tokyo Chemical Industry Co., Ltd.) were prepared.

Next, CHO cells introduced with an IgG expression gene (Mab A) were seeded in a 250 mL baffled Erlenmeyer flask containing the prepared production medium, and cultured with shaking in a CO₂ incubator for 13 days. The catechin hydrate-containing feed medium was added such that a catechin hydrate concentration in the culture medium at the end of the culture was 0 μmol/L, 122.3 μmol/L, or 305.9 μmol/L, or the catechin hydrate-free feed medium was added, followed by performing fed-batch culture. Other various culture conditions thereof were Mab A highly-productive process 2 conditions (Table 1). An antibody concentration was measured using a Protein A HPLC.

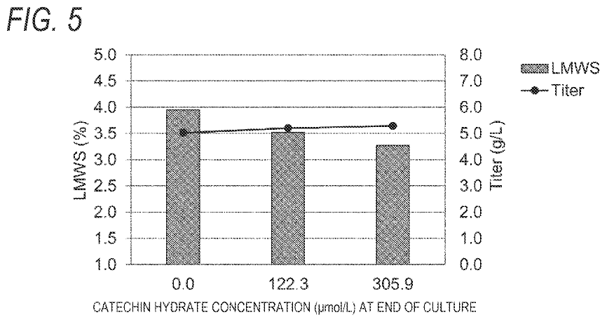

A produced antibody (Mab A) was affinity-purified from the culture medium on day 13 of culture using a Protein A resin, and subjected to capillary electrophoresis under non-reduction conditions using a Labchip GXII touch HT (manufactured by PerkinElmer Co., Ltd.) to evaluate an LMWS amount. The results are shown in FIG. 5.

As shown in FIG. 5, it was confirmed that the LMWS amount of the produced antibody was 4.0% under a condition where the catechin hydrate-free feed medium was added, was reduced to 3.5% under a condition where the catechin hydrate concentration was 122.3 μmol/L, and was reduced to 3.3% under a condition where the catechin hydrate concentration was 305.9 μmol/L.

As shown in FIG. 5, the produced antibody concentration was 5.0 g/L under a condition where the catechin hydrate-free feed medium was added, whereas the produced antibody concentration was 5.2 g/L under a condition where the catechin hydrate concentration was 122.3 μmol/L, and was 5.3 g/L under a condition where the catechin hydrate concentration was 305.9 μmol/L, and no reduction was observed.

From the above, it has become clear that the LMWS amount can be reduced while the antibody production amount can be maintained by adding catechin hydrate into the medium under highly-productive process conditions.

### Example 5

It has become clear that an LMWS was significantly reduced as a quality of a produced antibody when epigallocatechin gallate or catechin hydrate having an antioxidant action was added to a medium used for cell culture in a highly-productive process.

A culture test was performed using a 2 L glass reactor. Preparation of a medium and culture were performed by the following procedure. First, feed media with epigallocatechin gallate (Catalog No.: 02566-76, manufactured by Nacalai Tesque, Inc.) or catechin hydrate (Catalog No.: C0705, manufactured by Tokyo Chemical Industry Co., Ltd.) and without either were prepared.

Next, CHO cells introduced with an IgG expression gene (Mab A) were seeded in a 2 L glass reactor containing the prepared production medium and cultured for 13 days. The epigallocatechin gallate-containing feed medium or the catechin hydrate-containing feed medium was added such that an epigallocatechin gallate concentration in the culture medium at the end of the culture was 77.5 μmol/L or a catechin hydrate concentration in the culture medium at the end of the culture was 122.3 μmol/L, followed by performing fed-batch culture. Culture was performed in parallel using the feed medium containing neither epigallocatechin gallate nor catechin hydrate. Other various culture conditions thereof were Mab A highly-productive process 2 conditions. The culture under each condition was performed at n=3.

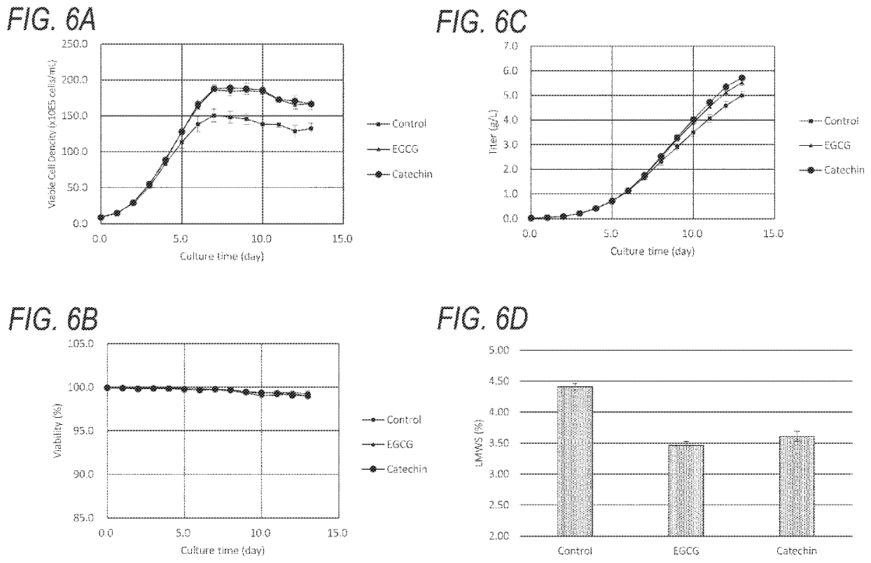

A viable cell density and viability during a culture time were measured using a Vi-CELL XR (manufactured by Beckman Coulter, Inc.). The results are shown in FIGS. 6A and 6B.

As shown in FIGS. 6A and 6B, the viable cell density remained higher under a condition where epigallocatechin gallate or catechin hydrate was added than under a condition where epigallocatechin gallate or catechin hydrate was not added. On the other hand, no difference was observed in the viability. When epigallocatechin gallate or catechin hydrate is added to a medium for culture, it has been known, for example, in WO 2014/182658 or the like that the viable cell density and the viability are reduced in the case where the addition amount of epigallocatechin gallate or catechin hydrate reaches a high concentration. However, it was confirmed that there is no problem with the effect on proliferation within the concentration range of this addition.

A produced antibody (Mab A) was affinity-purified from the culture medium on day 13 of culture using a Protein A resin, and subjected to capillary electrophoresis under non-reduction conditions using a Labchip GXII touch HT (manufactured by PerkinElmer Co., Ltd.) to evaluate an LMWS amount. The results are shown in FIG. 6D.

As shown in FIG. 6D, it was confirmed that an average LMWS amount of the produced antibody was 4.5% under a condition where the feed medium containing neither epigallocatechin gallate nor catechin hydrate was added, whereas the average LMWS amount in the produced antibody was 3.4% under a condition where epigallocatechin gallate was added and 3.6% under a condition where catechin hydrate was added, which was a significant reduction.

As shown in FIG. 6C, an average produced antibody concentration was 5.0 g/L under the condition where the feed medium containing neither epigallocatechin gallate nor catechin hydrate was added, whereas the average produced antibody concentration was 5.5 g/L under the condition where the epigallocatechin gallate was added and 5.7 g/L under the condition where the catechin hydrate was added, and an increase was observed.

From the above, it was also confirmed in a culture test using a 2 L glass reactor that by adding epigallocatechin gallate or catechin hydrate in a medium under highly-productive process conditions, the LMWS amount can be significantly reduced while the antibody production amount can be maintained.

### [Example 6] LMWS Reduction Effect by Cystine Concentration Reduction

An effect of changing a cystine concentration in a medium used for cell culture on quality of an antibody produced in a highly-productive process was studied, and it was found that an LMWS was reduced.

Preparation of a medium and culture were performed by the following procedure. First, feed media with different cystine concentrations were prepared by adding cystine (C6727-1 kg, manufactured by Sigma-Aldrich) during feed medium preparation.

Next, CHO cells (Mab A) introduced with an IgG expression gene were seeded in a 250 mL baffled Erlenmeyer flask containing the prepared production medium, and cultured with shaking in a CO₂ incubator for 13 days. The feed media with different cystine concentrations were added such that the cystine concentrations in the culture medium at the end of the culture were 1.20 mmol/L, 1.43 mmol/L, 1.66 mmol/L, 1.89 mmol/L, and 2.12 mmol/L, followed by fed-batch culture. Other various culture conditions thereof were Mab A highly-productive process 3 conditions. An antibody concentration was measured using a Protein A HPLC.

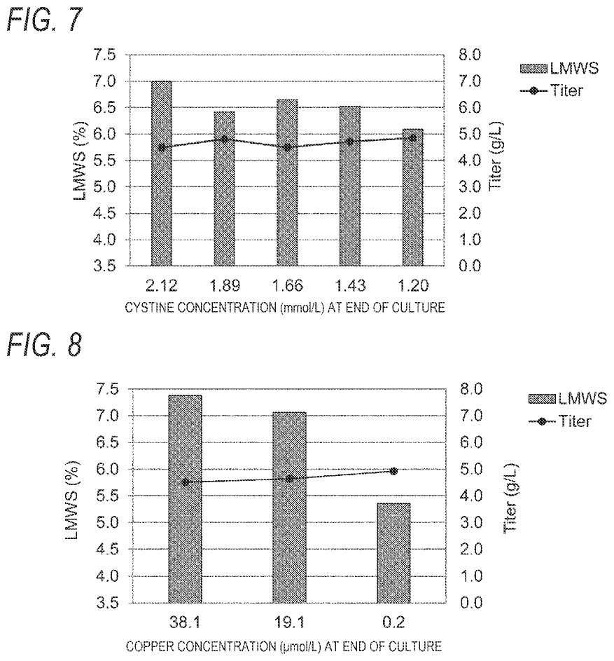

A produced antibody (Mab A) was affinity-purified from the culture medium on day 13 of culture using a Protein A resin, and subjected to capillary electrophoresis under non-reduction conditions using a Labchip GXII touch HT (manufactured by PerkinElmer Co., Ltd.) to evaluate an LMWS amount. The results are shown in FIG. 7.

As shown in FIG. 7, the LMWS amount of the produced antibody tended to be reduced as the cystine concentration was reduced, and on the other hand, the antibody concentration did not change.

From the above, it has become clear that the LMWS amount can be reduced while the antibody production amount can be maintained by reducing the cystine concentration in the culture medium under highly-productive process conditions.

### [Example 7] LMWS Reduction Effect by Copper Concentration Reduction

An effect of changing a copper concentration in a medium used for cell culture on quality of an antibody produced in a highly-productive process was studied, and it was found that an LMWS was reduced.

Preparation of a medium and culture were performed by the following procedure. First, production media with different copper concentrations were prepared by adding copper (II) sulfate pentahydrate (Catalog No.: 039-04412, manufactured by FUJIFILM Wako Pure Chemical Corporation) during production medium preparation.

Next, CHO cells introduced with an IgG expression gene (Mab A) were seeded in 250 mL baffled Erlenmeyer flasks containing various prepared production medium, and cultured with shaking in a CO₂ incubator for 17 days. During a culture time, a feed medium was added, and a fed-batch culture was performed such that the copper concentration in the culture medium at the end of the culture was 0.2 μmol/L, 19.1 μmol/L, or 38.1 μmol/L. Other various culture conditions thereof were Mab A highly-productive process 2 conditions. An antibody concentration was measured using a Protein A HPLC.

A produced antibody (Mab A) was affinity-purified from the culture medium on day 13 of culture using a Protein A resin, and subjected to capillary electrophoresis under non-reduction conditions using a Labchip GXII touch HT (manufactured by PerkinElmer Co., Ltd.) to evaluate an LMWS amount. The results are shown in FIG. 8.

As shown in FIG. 8, the LMWS amount of the produced antibody tended to be reduced as the copper concentration was reduced, and on the other hand, the antibody concentration did not change.

From the above, it has become clear that the LMWS amount can be reduced while the antibody production amount can be maintained by reducing the copper concentration in the culture medium under highly-productive process conditions.

### [Example 8] LMWS Reduction Effect by Citric Acid Addition

An effect of adding citric acid in a medium used for cell culture on quality of an antibody produced in a highly-productive process was studied, and it was found that an LMWS was reduced.

Preparation of a medium and culture were performed by the following procedure. First, production media with different citric acid concentrations were prepared by adding sodium citrate (manufactured by Kozakai Pharmaceutical Co., Ltd., Japanese pharmacopeia) during production medium preparation. Feed media with different citric acid concentrations were prepared by adding sodium citrate (manufactured by Kozakai Pharmaceutical Co., Ltd., Japanese pharmacopeia) during feed medium preparation.

Next, CHO cells introduced with an IgG expression gene (Mab A) were seeded in 250 mL baffled Erlenmeyer flasks containing various prepared production medium, and cultured with shaking in a CO₂ incubator for 17 days. The feed media were added such that the citric acid concentrations in the culture medium at the end of the culture was 0.14 mmol/L, 1.84 mmol/L, 2.06 mmol/L, 3.97 mmol/L, 5.24 mmol/L, and 5.89 mmol/L, followed by fed-batch culture. Other various culture conditions thereof were Mab A highly-productive process 2 conditions. An antibody concentration was measured using a Protein A HPLC.

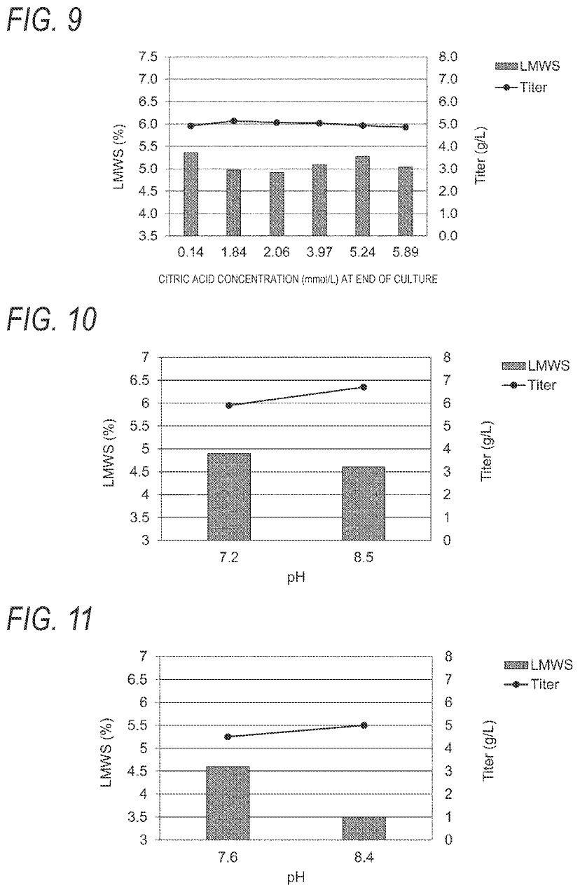

A produced antibody (Mab A) was affinity-purified from the culture medium on day 13 of culture using a Protein A resin, and subjected to capillary electrophoresis under non-reduction conditions using a Labchip GXII touch HT (manufactured by PerkinElmer Co., Ltd.) to evaluate an LMWS amount. The results are shown in FIG. 9.

As shown in FIG. 9, the LMWS amount of the produced antibody was reduced when the citric acid concentration is 1.84 mmol/L or more, and on the other hand, the antibody concentration did not change.

From the above, it has become clear that the LMWS amount can be reduced while the antibody production amount can be maintained by increasing the citric acid concentration in the culture medium under highly-productive process conditions.

### [Example 9] LMWS Reduction Effect by pH Increase of Medium

An effect of increasing a pH of a medium used for cell culture on quality of an antibody produced in a highly-productive process was studied, and it was found that an LMWS was reduced.

Preparation of a medium and culture were performed by the following procedure. First, a pH of a feed medium was adjusted to 7.2 and 8.5. Next, CHO cells introduced with an IgG expression gene (Mab B) were seeded in a 2 L glass reactor containing the prepared production medium and cultured for 14 days. Other various culture conditions thereof were Mab B highly-productive process conditions. During a culture time, the feed medium was added. An antibody concentration was measured using a Protein A HPLC.

A produced antibody (Mab B) was affinity-purified from a culture medium on day 14 of culture using a Protein A resin, and subjected to capillary electrophoresis using a Proteome Lab PA 800 plus (manufactured by AB Sciex) to evaluate an LMWS amount. The results are shown in FIG. 10.

As shown in FIG. 10, the LMWS amount of the produced antibody was 4.9% when the pH of the feed medium was 7.2, whereas the LMWS amount in the produced antibody was 4.6% when the pH of the feed medium was 8.5.

As shown in FIG. 10, the produced antibody concentration was 5.9 g/L when the pH of the feed medium was 7.2, and increased to 6.7 g/L when the pH of the feed medium was 8.5.

Next, an effect of increasing a pH of a medium used for cell culture on quality of an antibody produced in a highly-productive process was also studied for Mab C-producing CHO cells. Preparation of a medium and culture were performed by the following procedure. First, a pH of a feed medium was adjusted to 7.6 and 8.4.

Next, CHO cells introduced with an IgG expression gene (Mab C) were seeded in a 3 L glass reactor filled with the prepared production medium and cultured for 13 days. Other various culture conditions thereof were Mab C highly-productive process conditions. During a culture time, the feed medium was added. An antibody concentration was measured using a Protein A HPLC.

A produced antibody (Mab C) was affinity-purified from a culture medium on day 13 of culture using a Protein A resin, and subjected to capillary electrophoresis using a Proteome Lab PA 800 plus (manufactured by AB Sciex) to evaluate an LMWS amount. The results are shown in FIG. 11.

As shown in FIG. 11, the LMWS amount of the produced antibody was 4.6% when the pH of the feed medium was 7.6, whereas the LMWS amount in the produced antibody was 3.5% when the pH of the feed medium was 8.4.

As shown in FIG. 11, the produced antibody concentration was 4.5 g/L when the pH of the feed medium was 7.6, and increased to 5.0 g/L when the pH of the feed medium was 8.4.

From the above, it has become clear that the LMWS amount can be reduced while the antibody production amount can be maintained at the same level or higher by increasing the pH of the medium under highly-productive process conditions.

Although the present invention has been described in detail with reference to specific aspects, it is obvious to those skilled in the art that various changes and modifications can be made without departing from the spirit and scope of the present invention. The present application is based on Japanese patent application JP 2021-073666 filed on Apr. 23, 2021, the disclosure of which is hereby incorporated by reference herein in its entirety. All references cited herein are incorporated in their entirety.

1. A method for preventing generation of a degradation product (a low molecular weight species: LMWS) of a target protein, the method comprising: producing the target protein at a high concentration in a culture medium during a cell culture process, and removing a reactive oxygen species in the culture medium during said cell culture process.

2. The method according to claim 1, wherein the target protein is an antibody, and an antibody concentration in the culture medium at the end of the cell culture is 4.0 g/L or more.

3. The method according to claim 12, wherein the amount of the LMWS generated is reduced as compared with a cell culture process not including the removing of the reactive oxygen species.

4-6. (canceled)

7. The method according to claim 1, wherein the removing of the reactive oxygen species includes at least one selected from the following (a) to (e):
(a) adding an antioxidant to a medium used for the cell culture,
(b) making a cystine or cystine analogue concentration in the culture medium at the end of the cell culture 1.90 mmol/L or less,
(c) making a copper concentration in the culture medium at the end of the cell culture 20.0 μmol/L or less,
(d) adding a chelating compound to a medium used for the cell culture, and
(e) making a pH of a feed medium used for the cell culture 8.0 or more.

8. The method according to claim 7, wherein in the (a), the antioxidant is a catechin analogue.

9. The method according to claim 8, wherein a catechin analogue concentration in the culture medium at the end of the cell culture is 50 μmol/L or more.

10-13. (canceled)

14. The method according to claim 7, wherein in (b), the cystine or cystine analogue concentration in the culture medium at the end of the cell culture is 0.50 mmol/L to 1.90 mmol/L.

15. (canceled)

16. The method according to claim 7, wherein in (c), the copper concentration in the culture medium at the end of the cell culture is 0.10 μmol/L to 20.0 μmol/L.

17. (canceled)

18. The method according to claim 7, wherein in (d), the chelating compound is EDTA or citric acid.

19-20. (canceled)

21. The method according to claim 7, wherein in (e), the pH of the feed medium is 8.0 to 9.0.

22. (canceled)

23. A method for producing a target protein with a reduced LMWS amount at a high concentration in a culture medium, the method comprising: removing a reactive oxygen species in the culture medium during a cell culture process.

24. The method according to claim 23, wherein the target protein is an antibody, and an antibody concentration in the culture medium at the end of the cell culture is 4.0 g/L or more.

25. The method according to claim 2324, wherein the amount of the LMWS generated is reduced as compared with a cell culture process not including the removing of the reactive oxygen species.

26-28. (canceled)

29. The method according to claim 23, wherein the removing of the reactive oxygen species includes at least one selected from the following (a) to (e):
(a) adding an antioxidant to a medium used for the cell culture,
(b) making a cystine or cystine analogue concentration in the culture medium at the end of the cell culture 1.90 mmol/L or less,
(c) making a copper concentration in the culture medium at the end of the cell culture 20.0 μmol/L or less,
(d) adding a chelating compound to a medium used for the cell culture, and
(e) making a pH of a feed medium used for the cell culture 8.0 or more.

30. The method according to claim 29, wherein in (a), the antioxidant is a catechin analogue.

31. The method according to claim 30, wherein a catechin analogue concentration in the culture medium at the end of the cell culture is 50 μmol/L or more.

32-35. (canceled)

36. The method according to claim 29, wherein in (b), the cystine or cystine analogue concentration in the culture medium at the end of the cell culture is 0.50 mmol/L to 1.90 mmol/L.

37. (canceled)

38. The method according to claim 29, wherein in (c), the copper concentration in the culture medium at the end of the cell culture is 0.10 μmol/L to 20.0 μmol/L.

39. (canceled)

40. The method according to claim 29, wherein in (d), the chelating compound is EDTA or citric acid.

41-42. (canceled)

43. The method according to claim 29, wherein in (e), the pH of the feed medium is 8.0 to 9.0.

44. (canceled)
